(19) United States
(12) Patent Application Publication
(10) Pub. No.: US 2016/0340720 A1
(43) Pub. Date: Nov. 24, 2016

(54) METHODS AND COMPOSITIONS FOR ARRAY-BASED COUNTING

(71) Applicant: CELLULAR RESEARCH, Palo Alto, CA (US)

(72) Inventors: Christina Fan, Fremont, CA (US); Glenn FU, Dublin, CA (US); Julie Wilhelmy, Santa Cruz, CA (US)

(21) Appl. No.: 15/108,268

(22) PCT Filed: Feb. 13, 2015
(86) PCT No.: PCT/US15/15967

§ 371 Date: Jun. 24, 2016

Related U.S. Application Data
(60) Provisional application No. 61/939,497, filed on Feb. 13, 2014.

(51) Int. Cl. C12Q 1/68

(52) U.S. Cl. CPC C12Q 1/6816

(57) ABSTRACT

Methods, kits, and systems are disclosed for array-based counting. The method generally comprises obtaining data pertaining to one or more probes on an array and conducting probe-specific analysis of the data to determine a count of one or more objects hybridized to the probe on the array. The probe-specific analysis may comprise modeling a behavior of one or more probes. The objects hybridized to the probe may be nucleic acids from a sample. The sample may comprise a total quantity of nucleic acids of less than one genome equivalent. The array may be a tiling array. The methods, kits, and systems may be used for quantifying samples comprising low quantities of nucleic acids. The methods, kits, and systems may be used to diagnose a disease or condition in a subject. The methods, kits, and systems may be used to diagnose a fetal disorder.

### CROSS-REFERENCE

This application claims the benefit of U.S. Provisional Application No. 61/939,497, filed Feb. 13, 2014, which application is incorporated herein by reference.

### BACKGROUND OF THE INVENTION

Array technologies have been widely used in biomedical studies. Arrays are equipped with probes which can be hybridized with target molecules with labels, e.g., fluorescence. A feature on an array is a small cluster of the same or similar probes with specific molecular sequences, say, DNA or RNA. Identifying the label patterns on a hybridized array can infer the hybridization taking place in the sample, which in turn can further assist biomedical studies. Two important engineering steps prior to performing biomedical investigations are (i) to image the hybridized arrays and (ii) to analyze the images. Existing systems of image formation usually take the image of a region on an array at a time, which is a slow process when a number of regions have to be imaged. On the other hand, current methods of image analysis merely summarize an intensity level (i.e., an analog quantity) for a feature. Nevertheless, the intensity levels cannot truly reflect the presence or absence of labels attached onto features, but the determination of present labeled features frequently is a desired outcome in many biomedical studies. The limitations sometimes prohibit the use of the array technologies to advance biomedical discoveries.

### SUMMARY OF THE INVENTION

Disclosed herein are methods of determining a count of one or more nucleic acids, the method comprising (a) hybridizing a plurality of nucleic acids from a sample to one or more probes on an array to produce one or more hybridized nucleic acids, wherein the plurality of nucleic acids may comprise one or more molecular species; (b) scanning the array to obtain a detection signal for the one or more probes; (c) converting the detection signal to a digital signal; and (d) determining a count of the one or more hybridized nucleic acids based on a count of the digital signal.

Further disclosed herein are methods of determining a count of one or more nucleic acids, the method comprising (a) hybridizing a plurality of nucleic acids to one or more probes on an array to produce one or more hybridized nucleic acids; (b) detecting the one or more probes on the array to obtain a detection signal of the one or more probes; and (c) determining a count of the one or more hybridized nucleic acids by applying an algorithm to the detection signal.

Further disclosed herein are methods of determining a count of one or more nucleic acids, the method comprising (a) obtaining a detection signal for one or more probes on an array; (b) conducting one or more probe-specific analyses on the detection signal; and (c) determining a count of one or more nucleic acids based on results from the one or more probe-specific analyses.

Further disclosed herein are methods of determining a count of one or more molecules, the method comprising (a) hybridizing a plurality of molecules to a plurality of probes on an solid support to produce one or more hybridized molecules; and (b) determining a count of the one or more hybridized molecules by conducting one or more probe-specific analyses on the one or more probes.

Further disclosed herein are methods of determining a count of one or more molecules comprising (a) hybridizing a plurality of molecules to one or more probes on an array to produce one or more hybridized molecules; (b) counting individual probes on the array; (c) classifying the individual probes on the array based on the detection of the individual probes; and (d) determining a count of one or more molecules based on the classification of the individual probes.

Further disclosed herein are methods of determining a count of one or more molecules comprising (a) hybridizing a plurality of molecules to one or more probes on an array to produce one or more hybridized molecules; and (b) counting individual probes on the array, thereby determining a count of one or more molecules, wherein the method does not comprise labeling the plurality of molecules with barcodes, wherein the barcodes are used to differentiate two or more molecules of the same molecular species.

Further disclosed herein are methods of prenatal diagnostics. Generally, the method comprises (a) hybridizing a plurality of molecules from a sample from pregnant subject to one or more probes on a solid support; (b) conducting one or more probe-specific analyses of the one or more probes on the solid support; and (c) diagnosing, predicting, or monitoring a status or outcome of a fetal condition of a fetus in the pregnant subject based on results of the one or more probe-specific analyses.

Further disclosed herein are methods of prenatal diagnostics, the method comprising (a) hybridizing a plurality of nucleic acids from a sample from a pregnant subject to one or more probes on an array to produce one or more hybridized nucleic acids, wherein the plurality of nucleic acids may comprise one or more molecular species; (b) scanning the array to obtain a detection signal for the one or more probes; (c) converting the detection signal to a digital signal; (d) determining a count of the one or more hybridized nucleic acids based on a count of the digital signal; and (e) diagnosing, predicting, or monitoring a status or outcome of a fetal condition of a fetus in the pregnant subject based on the count of the one or more hybridized nucleic acids.

Further disclosed herein are methods of prenatal diagnostics, the method comprising (a) hybridizing a plurality of nucleic acids to one or more probes on an array to produce one or more hybridized nucleic acids; (b) detecting the one or more probes on the array to obtain a detection signal of the one or more probes; (c) determining a count of the one or more hybridized nucleic acids by applying an algorithm to the detection signal; and (d) diagnosing, predicting, or monitoring a status or outcome of a fetal condition of a fetus in the pregnant subject based on the count of the one or more hybridized nucleic acids.

Further disclosed herein are kits for array-based quantification. Generally, the kits for use in array-based quantification comprise a software program comprising computer-executable instructions for converting a detection signal from one or more probes to a digital signal. The kit for use in array-based quantification may comprise a software program comprising computer-executable instructions for conducting one or more probe-specific analyses of one or more probes on an array. The kit for use in array-based quantification may comprise a software program comprising computer-executable instructions for modeling probe behavior based on one or more probe features. The kit for use in array-based quantification may comprise a software program comprising computer-executable instructions for modeling probe behavior based on probe sequence. The kit for use in array-based quantification may comprise a software program comprising computer-executable instructions for modeling probe behavior using multivariate linear regression. The kits may further comprise a solid support comprising one or more probes.

Further disclosed herein are systems for array-based quantification. Generally, the systems comprise (a) a solid support comprising one or more probes; and (b) a computer readable medium comprising instructions for converting a detection signal from one or more probes to a digital signal.

The computer-implemented system may comprise (a) a digital processing device comprising an operating system configured to perform executable instructions and a memory device; and (b) a computer program including instructions executable by the digital processing device to determine a count of one or more nucleic acids hybridized to an array comprising (i) a software module configured to receive data input pertaining to one or more probes on the array; (ii) a software module configured to conduct probe-specific analysis of the data input and to generate an output based on the probe-specific analysis; and (iii) a software module configured to generate an output of the count of the one or more nucleic acid molecules hybridized to the array.

The computer readable medium may comprise instructions for detecting 50 or more different probes on the solid support. The computer readable medium may comprise instructions for detecting 40,000 or more probes on the solid support. The computer readable medium may comprise instructions for detecting 50,000 or more probes on the solid support.

Disclosed herein are methods, kits, and systems for solid support-based digital quantification of molecules. The molecules may be nucleic acids. The nucleic acids may be DNA. The nucleic acids may be RNA. The nucleic acids may be hybridized to one or more probes on a solid support. The nucleic acids that are hybridized to the one or more probes on the solid support may be referred to as hybridized nucleic acids or probe hybridized nucleic acids.

Determining the count of the one or more molecules may comprise determining an intensity value for one or more individual probes on a solid support. The intensity value for an individual probe may be based on an image of the probe(s). The image of the probe(s) may be obtained by using a detection system. The detection system may comprise a microscope. The detection system may comprise a macroscope. The detection system may comprise a fluorescence detection system. The detection system may comprise a scanner. The detection system may comprise a focus imaging system. The detection system may comprise a light source. The detection system may comprise a camera. The detection system may comprise an array reader.

Determining an intensity value for one or more individual probes on the solid support may comprise analysis of one or more images of the probes. Analysis of the images of the probes may comprise one or more statistical analyses on pixel measurements of the probes. The methods, software, kits, and systems disclosed herein may comprise a software module for performing one or more statistical analyses on pixel measurements to define dynamic thresholds on one or more regions on the solid support. A probe may correspond to a small area of bright pixels, and the background in the image may comprise purely dark pixels. In this ideal case, by way of a non-limiting example, bright pixels may have measurements as 1 and dark pixels as 0 in an image, and a threshold can be arbitrarily determined, e.g., any quantity larger than 0 and smaller than 1, to classify where the bright and dark pixels may locate. In cases in which various sources of noise may make the bright pixels not so bright and the dark pixels not so dark, an alternative method of classifying a probe may comprise statistically analyzing images of the probes.

In the image analysis, a small window of, by way of non-limiting examples, 5×5, 7×7, 9×9, 15×15, 51×51, or 101×101 pixels, centered at a probe may be defined. A threshold associated to a probe may be determined by statistical analyses on the pixel measurements within the window. The threshold may change from probe to probe, and it can be dynamically derived for different probes by analyzing pixel measurements within their windows. Producing the dynamic threshold for a probe may comprise comparing pixel measurements in the background and foreground areas. Alternatively, the foreground and background pixel measurements can be described by different mathematical models or probabilistic distributions, and then suitable analyses may be implemented to derive an optimal threshold. By way of non-limiting examples, suitable mathematical models may be K-means, probe reference distribution method, support vector machines, wavelet transforms, mixture models, graphical models, machine learning schemes, artificial intelligent methods, or expert systems, or a combination of the same. Furthermore, more sophisticated analyses may utilize more spatial and temporal information across windows, across regions, or across data collection time, or a combination of the same. In some cases, another source of information (by way of non-limiting examples, locations of probes, frequently occurred artifact patterns, human knowledge, human guidance, previously derived results, literature report, solid support manufacturers' suggestions) may be integrated into the analysis.

The methods, kits, and systems disclosed herein may be used to detect one or more probes within one or more regions on a solid support. Detecting a probe within a region may comprise comparing the background-adjusted pixel measurements within a window with the dynamic threshold derived in the window. When a pixel measurement is above the threshold, the pixel may be classified as a labeled pixel, and a pixel may be classified as non-labeled if its measurement is below the threshold. Detecting a probe within a window may comprise determining if the classified labeled pixels form a cluster. By a non-limiting example, identifying a cluster of labeled pixels may be achieved by computationally examining the intensities of the labeled pixels have uniform distribution. Following the cluster identification may be a binary classification to discriminate the probe is labeled (“on”) or non-labeled (“off”). Those of skill in the art will recognize the variations of the steps and the possibility of interchanging the steps.

The methods, software, kits, and systems disclosed herein may comprise counting one or more probes within one or more regions on a solid support. Once probes have been detected, counting the number of probes may be equivalent to counting the number of classified “on” within the region. In some applications, the probes may be represented by “off,” where counting the number of probes may become counting the number of “off” in the region.

Determining the count of the one or more molecules may further comprise classifying the probes as positive or negative. The probes may be classified as positive if the intensity value of the probes is greater than a background intensity value. The probes may be classified as negative if the intensity value of the probes is less than a background intensity value. Determining the count of the one or more molecules may further comprise determining a count of probes that are classified as positive.

Determining the count of the one or more molecules may comprise use of a binary classification system. The binary classification system may be used to classify one or more probes on the solid support. Binary classification of the one or more probes may be based on a signal from the probe. The signal may be an analogue signal. The binary classification system may convert an analogue signal of the probe to a digital signal.

Determining the count of the one or more hybridized nucleic acids may comprise converting the analog signal to a digital signal. Converting the detection signal to a digital signal may comprise binary classification of the detection signal. The binary classification may comprise classifying a probe as positive or negative. Determining the count of the one or more hybridized nucleic acids may comprise determining a count of probes that are classified as positive.

Determining the count of the one or more hybridized nucleic acids may comprise modeling probe behavior of the one or more probes. Converting the detection signal to a digital signal may comprise modeling a behavior of the one or more probes based on the sequence of the probe.

Modeling the behavior of the one or more probes may comprise applying an algorithm to the detection signal. The algorithm may be a multivariate linear regression. Modeling the behavior of the one or more probes may comprise subtracting a probe-specific background from the detection signal of the probe. Modeling the behavior of the one or more probes may comprise clustering the probes into groups based on similarity of one or more features. The one or more features may comprise GC content, probe length, and sequence alignment.

Determining the count of the one or more hybridized nucleic acids may comprise calculating a t score for the one or more probes on the array. Determining the count of the one or more hybridized nucleic acids further may comprise grouping the one or more probes into one or more regions. Determining the count of the one or more hybridized nucleic acids further may comprise calculating a trimmed mean t score for the one or more regions based on an average t score of the one or more probes in the one or more regions. Calculating the trimmed mean t score for the one or more regions may comprise (i) sorting the t score of the one or more probes within the one or more regions; (ii) discarding the t scores in the upper and lower 10% of the one or more probes within the one or regions; and (iii) calculating an average t score of the remaining probes within the one or more regions.

Determining the count of the one or more hybridized nucleic acids further may comprise calculating a model-based analysis of tiling arrays score (MATscore) of the one or more probes within the one or more regions by multiplying the square root of the number of probes within the region by the trimmed mean t score of the region.

The method further may comprise (a) hybridizing one or more additional pluralities of nucleic acids to one or more probes on one or more additional arrays to produce one or more hybridized nucleic acids; (b) detecting the one or more probes on the one or more additional arrays to obtain a detection signal of the one or more probes; and (c) determining a count of the one or more hybridized nucleic acids by applying an algorithm to the detection signal.

Determining the count of the one or more hybridized nucleic acids further may comprise calculating a corrected MATscore of the one or more probes by (i) calculating a median MATscore by averaging the MATscore of the one or more probes from the arrays; and (ii) subtracting the median MATscore from the MATscore, thereby calculating the corrected MATscore.

Determining the count of the one or more hybridized nucleic acids further may comprise classifying a probe as positive or negative based on the MATscore.

Determining the count of the one or more hybridized nucleic acids further may comprise counting the positive probes and correlating the count of the probes to the count of the one or more hybridized nucleic acids.

Determining the count of the one or more hybridized nucleic acids further may comprise classifying a probe as positive or negative based on the corrected MATscore.

Determining the count of the one or more hybridized nucleic acids further may comprise counting the positive probes and correlating the count of the probes to the count of the one or more hybridized nucleic acids.

The probe-specific analysis may comprise modeling a behavior of a probe based on a sequence of the probe. The probe-specific analysis may comprise determining probe-specific background data. The probe-specific analysis may comprise clustering the probes on the array into groups based on similarity of one or more features. The one or more features may comprise GC content. The one or more features may comprise nucleotide sequence. The one or more features may comprise sequence homology. The one or more features may comprise probe length. The one or more features may comprise repetitive sequences. The one or more features may comprise arrangement of the probes on the array. The probe-specific analysis may comprise determining background values for specific probes or groups of probes. The probe-specific analysis may comprise adjusting a detection signal of a probe by subtracting a probe-specific background from the detection signal to produce an adjusted signal.

Detecting the one or more probes may comprise detecting one or more probes that are hybridized to a nucleic acid of the plurality of nucleic acids. Detecting the one or more probes may comprise detecting one or more probes that are not hybridized to a nucleic acid of the plurality of nucleic acids. The detection signal may be an analog signal. The analog signal may be an intensity signal.

Counting individual probes on the solid support may comprise detecting two or more individual probes. Counting individual probes on the solid support may comprise detecting 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more individual probes. Counting individual probes on the solid support may comprise detecting 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000 or more individual probes. Counting individual probes on the solid support may comprise detecting 50 or more individual probes. Counting individual probes on the solid support may comprise detecting 100 or more individual probes. Counting individual probes on the solid support may comprise detecting 200 or more individual probes. Counting individual probes on the solid support may comprise detecting 500 or more individual probes. Counting individual probes on the solid support may comprise detecting 1000 or more individual probes. Counting individual probes on the solid support may comprise detecting 10000 or more individual probes. Counting individual probes on the solid support may comprise detecting 30000 or more individual probes. Counting individual probes on the solid support may comprise detecting 40000 or more individual probes. Counting individual probes on the solid support may comprise detecting 50000 or more individual probes. Counting individual probes on the solid support may comprise detecting 60000 or more individual probes.

Counting individual probes on the solid support may comprise detecting two or more different individual probes. Counting individual probes on the solid support may comprise detecting 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more different individual probes. Counting individual probes on the solid support may comprise detecting 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000 or more different individual probes. Counting individual probes on the solid support may comprise detecting 5 or more different probes. Counting individual probes on the solid support may comprise detecting 10 or more different probes. Counting individual probes on the solid support may comprise detecting 20 or more different probes. Counting individual probes on the solid support may comprise detecting 30 or more different probes. Counting individual probes on the solid support may comprise detecting 50 or more different probes. Counting individual probes on the solid support may comprise detecting 100 or more different probes. Counting individual probes on the solid support may comprise detecting 150 or more different probes. Counting individual probes on the solid support may comprise detecting 200 or more different probes. Counting individual probes on the solid support may comprise detecting 250 or more different probes. Counting individual probes on the solid support may comprise detecting 300 or more different probes.

A plurality of probes may be detected simultaneously. The plurality of probes may comprise 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more probes. The plurality of probes may comprise 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000 or more probes.

A plurality of probes may be detected sequentially. The plurality of probes may comprise 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more probes. The plurality of probes may comprise 200, 300, 400, 500, 600, 700, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000 or more probes.

A plurality of probes may be detected at two or more different time points. A plurality of probes may be detected at 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more different time points. A plurality of probes may be detected at 5 different time points. A plurality of probes may be detected at 10 different time points. The time points may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more minutes apart. The time points may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more hours apart. The time points may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more days apart. The time points may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more weeks apart. The time points may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more months apart. The time points may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more years apart.

The methods disclosed herein may further comprise determining a count of the one or more molecular species based on the count of the one or more hybridized nucleic acids. The methods disclosed herein may further comprise determining a count of two or more molecular species based on the count of the one or more hybridized nucleic acids The two or more molecular species may be less than about 90% identical. The two or more molecular species may be greater than about 20% identical.

The one or more molecular species may comprise a genomic region or portion thereof. The one or more molecular species may comprise a chromosome or portion thereof. The chromosome may be chromosome 21. The chromosome may be chromosome 18. The chromosome may be chromosome 13. The chromosome may be an X chromosome. The chromosome may be a Y chromosome. The chromosome may be chromosome 22. The one or more molecular species may comprise a gene or portion thereof. The one or more molecular species may comprise an exon or portion thereof. The one or more molecular species may comprise an intron or portion thereof. The one or more molecular species may comprise an untranslated region or portion thereof. The one or more molecular species may comprise a protein coding region or portion thereof. The one or more molecular species may comprise a non-coding region or portion thereof. The one or more molecular species may comprise a breakpoint junction or portion thereof. The one or more molecular species may comprise a structural variant or portion thereof.

The methods disclosed herein may further comprise determining a copy number of one or more molecules. Determining a copy number of a molecule may comprise determining the relative copy number of the molecule. Determining the relative copy number of the molecule may comprise comparing a count of a first molecule to a count of a second molecule. Determining the copy number of a molecule may comprise determining the absolute copy number of the molecule.

The methods, kits, and systems disclosed herein may be used to quantify one or more molecules hybridized to one or more probes on a solid support. The molecules may be nucleic acids. The nucleic acids may be DNA. The DNA may be a cDNA. The nucleic acids may be RNA. The RNA may be mRNA. The RNA may be cRNA (e.g., antisense RNA). The RNA may be sense RNA. The nucleic acid may be double-stranded. The nucleic acid may be single-stranded. The nucleic acids may be cell-free nucleic acids. The nucleic acids may be circulating nucleic acids.

The method, kits, and systems disclosed herein may be used to determine a count of one or more nucleic acids from a sample. A total quantity of nucleic acids in the sample may be less than 1 genome equivalent. A total quantity of nucleic acids in the sample may be less than 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 genome equivalent. A total quantity of nucleic acids in the sample may be less than 1 haploid genome equivalent. A total quantity of nucleic acids in the sample may be less than 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 haploid genome equivalent.

A nucleic acid or product thereof may be less than about 500 basepairs in length. A nucleic acid or product thereof may be less than about 400 basepairs in length. A nucleic acid or product thereof may be less than about 300 basepairs in length. A nucleic acid or product thereof may be less than about 200 basepairs in length. A nucleic acid or product thereof may be less than about 190 basepairs in length. A nucleic acid or product thereof may be less than about 180 basepairs in length. A nucleic acid or product thereof may be less than about 170 basepairs in length.

A nucleic acid or product thereof may be greater than about 10 basepairs in length. A nucleic acid or product thereof may be greater than about 20 basepairs in length. A nucleic acid or product thereof may be greater than about 30 basepairs in length. A nucleic acid or product thereof may be greater than about 40 basepairs in length. A nucleic acid or product thereof may be greater than about 50 basepairs in length.

A nucleic acid or product thereof may be between about 10 to about 500 basepairs in length. A nucleic acid or product thereof may be between about 10 to about 400 basepairs in length. A nucleic acid or product thereof may be between about 10 to about 200 basepairs in length. A nucleic acid or product thereof may be between about 30 to about 500 basepairs in length. A nucleic acid or product thereof may be between about 30 to about 400 basepairs in length. A nucleic acid or product thereof may be between about 30 to about 300 basepairs in length. A nucleic acid or product thereof may be between about 30 to about 200 basepairs in length. A nucleic acid or product thereof may be between about 40 to about 200 basepairs in length. A nucleic acid or product thereof may be between about 40 to about 170 basepairs in length.

Products of nucleic acids include, but are not limited to, nucleic acids that have been sheared, fragmented, transcribed, reverse transcribed, amplified, enriched, isolated, purified, ligated, hybridized, labeled, DNA end-labeled, polyA-tailed, and end-polished.

The methods disclosed herein may further comprise shearing one or more nucleic acids of the plurality of nucleic acids to produce one or more sheared nucleic acids. The one or more nucleic acids may be sheared prior to hybridization of the plurality of nucleic acids to the one or more probes on the array. Shearing the one or more nucleic acids may comprise mechanical shearing. Shearing the one or more nucleic acids may comprise sonicating. Shearing the one or more nucleic acids may comprise use of one or more restriction enzymes. The one or more restriction enzymes may comprise endonucleases. The one or more restriction enyzmes may comprise exonucleases. The one or more restriction enzymes may comprise methylation sensitive restriction enzymes.

The methods disclosed herein may further comprise end polishing the one or more nucleic acids to produce one or more end polished nucleic acids. The methods disclosed herein may further comprise end polishing of the one or more sheared nucleic acids to produce one or more end polished nucleic acids. End polishing may comprise the use of one or more end polishing enzymes.

The methods disclosed herein may further comprise polyA tailing the one or more nucleic acids to produce polyA tailed nucleic acids. The methods disclosed herein may further comprise polyA tailing the one or more end polished nucleic acids to produce one or more polyA tailed nucleic acids. PolyA tailing may comprise the use of one or more polyA tailing enzymes.

The methods disclosed herein may further comprise attaching one or more adaptors to the one or more nucleic acids or products thereof to produce adaptor modified nucleic acids. The adaptors may be attached to the nucleic acids by ligation. The adaptor may be attached to the nucleic acid by blunt end ligation. The adaptor may be attached to the nucleic acid by sticky end ligation. The adaptor may be attached to the nucleic acid by hybridization. The adaptor may be attached to the nucleic acid by transcription. The methods disclosed herein may further comprise ligating one or more adaptors to the one or more polyA tailed nucleic acids to produce one or more adaptor ligated nucleic acids.

The methods disclosed herein may further comprise estimating a concentration of the one or more nucleic acids or a product thereof. The methods disclosed herein may further comprise estimating a concentration of the one or more adaptor ligated nucleic acids. Estimating the concentration of the one or more adaptor ligated nucleic acids may comprise conducting a quantitative PCR reaction.

The methods disclosed herein may further comprise conducting an amplification reaction on the one or more nucleic acids or product thereof to produce one or more amplified nucleic acids. The methods disclosed herein may further comprise conducting an amplification reaction on the one or more adaptor ligated nucleic acids to produce one or more amplified nucleic acids. The methods disclosed herein may further comprise conducting an amplification reaction on the plurality of nucleic acids to produce one or more amplified nucleic acids, wherein the plurality of nucleic acids comprise one or more adaptor ligated nucleic acids or non-adaptor ligated nucleic acids.

The methods disclosed herein may further comprise fragmenting the one or more nucleic acids or products thereof to produce one or more fragmented nucleic acids. The methods disclosed herein may further comprise fragmenting the one or more amplified nucleic acids to produce one or more fragmented nucleic acids. Fragmenting the one or more nucleic acids may comprise use of one or more restriction enzymes.

The methods disclosed herein may further comprise labeling the one or more nucleic acids or products thereof to produce one or more labeled nucleic acids. The methods disclosed herein may further comprise labeling the one or more fragmented nucleic acids with one or more labels to produce one or more labeled nucleic acids. Labeling the one or more fragmented nucleic acids may comprise DNA end labeling. Labeling the one or more fragmented nucleic acids may comprise labeling with a terminal transferase. Labeling the one or more fragmented nucleic acids may comprise labeling with a biotinylated nucleotide.

Hybridizing the plurality of nucleic acids to the one or more probes on the array may comprise hybridizing the one or more nucleic acid products to the one or more probes on the array. Nucleic acid products may include, but may be not limited to, sheared nucleic acids, fragmented nucleic acids, adaptor-ligated nucleic acids, labeled nucleic acids, end-polished nucleic acids, polyA-tailed nucleic acids, enriched nucleic acids, amplified nucleic acids, and purified nucleic acids. Hybridizing the plurality of nucleic acids to the one or more probes on the array may comprise hybridizing the one or more labeled nucleic acids to the one or more probes on the array.

The methods disclosed herein may further comprise staining the one or more hybridized nucleic acids. Staining may comprise contacting the solid support with a detectable label. The detectable label may be a fluorophore. Examples of fluorophores include, but are not limited to, SYBR green, fluorescein, carboxyfluorescein, TET, rhodamin, coumarin, cyanine, FAM, FITC, DAPI, Cy5, Cy3, Texas Red, Alexa fluor 488, Alexa fluor 633, Alexa fluor 594, and Alexa fluor 568. The fluorophore may be Cy5. The fluorophore may be Cy3. The detectable label may be silver. The detectable label may be chemiluminescent label. The detectable label may be a chromophore.

The methods disclosed herein may further comprise scanning the array to individual probes on the array. The array may be scanned by using a detection system. The array may be scanned by a scanner. The array may be scanned by an array reader. Scanning may comprise use of a laser scanning instrument.

The methods, kits, and systems disclosed herein may comprise one or more probes or uses thereof. The one or more probes may selectively may hybridize to a genomic region associated with a disease or condition. The disease or condition may be a fetal disease or condition. The fetal disease or condition may be trisomy. The fetal disease or condition may be monosomy. The fetal disease or condition may be tetrasomy. The fetal disease or condition may be pentasomy. The fetal disease or condition may be monoploidy. The fetal disease or condition may be triploidy. The fetal disease or condition may be tetraploidy. The fetal disease or condition may be pentaploidy. The fetal disease or condition may be multiploidy.

The methods, kits, and systems disclosed herein may comprise one or more adaptors or uses thereof. The one or more adaptors may comprise a universal primer region. The one or more adaptors may comprise a sequencing primer region. The one or more adaptors comprise a barcode region.

The methods, kits, and systems disclosed herein may comprise one or more labels or uses thereof. The one or more labels may comprise a barcode region. The one or more labels may comprise a detectable label. The one or more labels may comprise an adaptor region. The one or more labels may comprise a universal primer region. The one or more labels may comprise a sequencing primer region.

The methods disclosed herein may further comprise diagnosing, predicting or monitoring a disease or condition in the subject. The disease or condition may be a fetal disease or condition. The disease or condition may be a cancer. The disease or condition may be pathogenic infection.

The methods disclosed herein may further comprise administering a therapeutic regimen to the subject. The methods disclosed herein may further comprise determining a therapeutic regimen.

The methods disclosed herein may comprise use of nucleic acids from a sample from a subject. The subject may be suffering from a disease or condition. The disease or condition may be a cancer. The disease or condition may be a pathogenic infection. The subject may be pregnant.

The methods disclosed herein may comprise nucleic acids from a sample. The sample may be a blood or plasma sample. The sample may be a urine sample. The sample may be a stool sample.

The methods, kits or systems disclosed herein may comprise an array comprising a plurality of probes or uses thereof. The plurality of probes may comprise a set of probes. The set of probes may be capable of hybridizing to one molecular species. The set of probes may be capable of hybridizing to one region on the one molecular species. The set of probes may be capable of hybridizing to two or more regions on the one molecular species. The set of probes may be capable of hybridizing to two molecular species.

The methods, kits and systems disclosed herein may comprise a software program or uses thereof. The software program may be provided as a computer-readable medium. The kits and systems disclosed herein may comprise instructions for installing a software program. The software program may comprise computer-executable instructions for digitally counting the one or more objects. The software program may comprise computer-executable instructions for scanning the array.

The software program may comprise computer-executable instructions for detecting the one or more objects hybridized to the one or more probes on the array. The software program may comprise computer-executable instructions for measuring an analogue signal emitted from the one or more objects (e.g., hybridized molecules). The software program may comprise computer-executable instructions for measuring an analogue signal emitted from the one or more probes. The analogue signal may be an intensity signal. The intensity signal may be a fluorescence intensity signal. The software program may comprise computer-executable instructions for converting the analogue signal to a digitized signal.

Converting the analogue signal to a digitized signal may comprise substracting a background analogue signal from the analogue signal emitted from the one or more objects to produce a background adjusted analogue signal. Converting the analogue signal may comprise comparing the background adjusted analogue signal to a control analogue value. The analogue signal may be converted to a countable digitized signal if the background adjusted analogue signal may be greater than the control analogue value. The analogue signal may be converted to a non-countable digitized signal if the background adjusted analogue signal may be less than the control analogue value. The instructions comprise computer-executable instructions for counting a number of countable digital signals, thereby digitally counting the one or more objects.

The methods, kits, and systems disclosed herein may comprise a detection system or uses thereof. The detection system may comprise a microscope. The detection system may comprise a macroscope. The detection system may comprise a fluorescence detection system. The detection system may comprise a scanner. The detection system may comprise a focus imaging system. The detection system may comprise a light source. The detection system may comprise a camera. The detection system may comprise an array reader.

The methods, kits, and systems disclosed herein may comprise a solid support or uses thereof. The solid support may be an array. The array may be a microarray. The microarray may be a tiling array.

The solid support may comprise one or more probes. The one or more probes may be tiling probes. The array may comprise 10, 20, 30, 40, 50, 60, 70, 80, 90, 100, 110, 120, 130, 140, 150, 160, 170, 180, 190, 200 or more different probes. The array may comprise 10 or more different probes. The array may comprise 20 or more different probes. The array may comprise 30 or more different probes. The array may comprise 40 or more different probes. The array may comprise 50 or more different probes. The array may comprise 75 or more different probes. The array may comprise 100 or more different probes. The array may comprise 125 or more different probes. The array may comprise 130 or more different probes. The array may comprise 140 or more different probes. The array may comprise 150 or more different probes. The array may comprise 200 or more different probes.

The array may comprise 40,000 or more probes. The array may comprise 45,000 or more probes. The array may comprise 50,000 or more probes. The array may comprise 60,000 or more probes. The array may comprise 70,000 or more probes. The array may comprise 80,000 or more probes. The array may comprise 90,000 or more probes. The array may comprise 100,000 or more probes. The array may comprise 110,000 or more probes. The array may comprise 120,000 or more probes.

At least two probes may be less than about 97%, 95%, 92%, 90%, 88%, 86%, 85%, 80%, 75%, 70%, 65%, 60%, 55%, or 50% identical. At least two probes may be less than about 90% identical. At least two probes may be less than about 80% identical. At least two probes may be less than about 60% identical. At least two probes may be less than about 50% identical. At least two probes may be less than about 40% identical. At least two probes may be less than about 30% identical. At least two probes may be less than about 25% identical.

At least two probes may be greater than about 1%, 2%, 3%, 4%, 5%, 6%, 7%, 8%, 9%, 10%, 12%, 14%, 15%, 17%, 20%, 22%, 25%, 27%, 30%, 32%, 35%, 37%, 40%, 45%, 50%, 55%, or 60% identical. At least two probes may be greater than about 1% identical. At least two probes may be greater than about 5% identical. At least two probes may be greater than about 10% identical. At least two probes may be greater than about 15% identical. At least two probes may be greater than about 20% identical.

At least two probes may be between about 10% to about 95% identical. At least two probes may be between about 20% to about 95% identical. At least two probes may be between about 20% to about 85% identical. At least two probes may be between about 20% to about 75% identical. At least two probes may be between about 25% to about 95% identical. At least two probes may be between about 30% to about 95% identical. At least two probes may be between about 25% to about 85% identical. At least two probes may be between about 25% to about 75% identical.

The one or more probes may hybridize to less than an entire genome. The one or more probes may hybridize to a portion of the genome. The one or more probes may hybridize to a chromosome. The one or more probes may hybridize to a portion of the chromosome. The one or more probes may hybridize to one or more regions within a chromosome. The one or more probes may hybridize two or more regions within a chromosome. At least two of the regions may be contiguous. At least two of the regions may be non-contiguous.

At least two probes may hybridize to at least two different genomic regions. The two different genomic regions overlap. The two different genomic regions overlap by at least about 5%. The two different genomic regions overlap by at least about 10%. The two different genomic regions overlap by at least about 20%.

At least about 5% of the plurality of probes may be overlapping probes. At least about 10% of the plurality of probes may be overlapping probes. At least about 20% of the plurality of probes may be overlapping probes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-B show bar graphs of the MATscore for genomic coordinates 1.6578-1.6584×10⁷ at 0.1x haploid genome (FIG. 1A) and 0.01x haploid genome (FIG. 1B).

FIG. 2A-B show bar graphs of the MATscore for genomic coordinates 9.884-9.89×10⁶ at 0.1x haploid genome (FIG. 2A) and 0.01x haploid genome (FIG. 2B).

FIG. 3A-D show bar graphs of the MATscore for genomic coordinates corresponding 4.228-4.23×10⁷ at 0.01x haploid genome (FIG. 3A-D). FIG. 3A shows the MATscore for the Normal 0.01x sample. FIG. 3B shows the MATscore for the 100% Trisomy 0.01x sample. FIG. 3C shows the MATscore for the 30% Trisomy 0.01x sample. FIG. 3D shows the MATscore for the 20% Trisomy 0.01x sample.

FIG. 4A-E show bar graphs of the MATscore for genomic coordinates corresponding 4.228-4.23×10⁷ at 0.01x haploid genome 0.1x haploid genome (FIG. 4A-E). FIG. 4A shows the MATscore for the Normal 0.1x sample. FIG. 4B shows the MATscore for the 100% Trisomy 0.1x sample. FIG. 4C shows the MATscore for the 30% Trisomy 0.1x sample. FIG. 4D shows the MATscore for the 20% Trisomy 0.1x sample. FIG. 4E shows the MATscore for the 10% Trisomy 0.1x sample.

FIG. 5A-D show bar graphs of the MATscore for genomic coordinates corresponding 4.912-4.914×10⁷ at 0.01x haploid genome (FIG. 5A-D). FIG. 5A shows the MATscore for the Normal 0.01x sample. FIG. 5B shows the MATscore for the 100% Trisomy 0.01x sample. FIG. 5C shows the MATscore for the 30% Trisomy 0.01x sample. FIG. 5D shows the MATscore for the 20% Trisomy 0.01x sample.

FIG. 6A-D show bar graphs of the MATscore for genomic coordinates corresponding 4.912-4.914×10⁷ at 0.1x haploid genome (FIG. 6A-E). FIG. 6A shows the MATscore for the Normal 0.1x sample. FIG. 6B shows the MATscore for the 100% Trisomy 0.1x sample. FIG. 6C shows the MATscore for the 30% Trisomy 0.1x sample. FIG. 6D shows the MATscore for the 20% Trisomy 0.1x sample.

FIG. 7A-D show bar graphs of the corrected MATscore for genomic coordinates 1.6578-1.6584×10⁷ at 0.01x haploid genome. FIG. 7A shows the corrected MATscore for the Normal 0.01x sample. FIG. 7B shows the corrected MATscore for the 100% Trisomy 0.01x sample. FIG. 7C shows the corrected MATscore for the 30% Trisomy 0.01x sample. FIG. 7D shows the corrected MATscore for the 20% Trisomy 0.01x sample.

FIG. 8A-D show bar graphs of the corrected MATscore for genomic coordinates 9.884-9.89×10⁶ at 0.01x haploid genome. FIG. 8A shows the corrected MATscore for the Normal 0.01x sample. FIG. 8B shows the corrected MATscore for the 100% Trisomy 0.01x sample. FIG. 8C shows the corrected MATscore for the 30% Trisomy 0.01x sample. FIG. 8D shows the corrected MATscore for the 20% Trisomy 0.01x sample.

FIG. 9A-B show bar graphs of the number of peaks over an entire chromosome (chromosomes 21 and 22) at 0.01x haploid genome (FIG. 9A) and 0.1x haploid genome (FIG. 9B).

FIG. 10A-B show graphs of the peak ratios for chromosome 21/22 for the 0.01x haploid concentration (FIG. 10A) and 0.1x haploid concentration (FIG. 10B).

FIG. 11A shows the names and descriptions of the samples used in example 4.

FIG. 11B shows a graph of the peak ratios for chromosome 21/22 using a MATscore cutoff of 2.5 for the samples described in FIG. 11A. The sample names are shown on the x-axis.

### DETAILED DESCRIPTION OF THE INVENTION

Disclosed herein are methods of determining a count of one or more molecules. Generally, the method comprises (a) hybridizing a plurality of nucleic acids from a sample to one or more probes on an array to produce one or more hybridized nucleic acids, wherein the plurality of nucleic acids may comprise one or more molecular species; (b) scanning the array to obtain a detection signal for the one or more probes; (c) converting the detection signal to a digital signal; and (d) determining a count of the one or more hybridized nucleic acids based on a count of the digital signal. The molecules may be nucleic acids. The nucleic acids may be DNA. The nucleic acids may be RNA. The solid support may be an array. The array may be a tiling array. The probes may comprise tiling probes.

Further disclosed herein are methods of determining a count of one or more nucleic acids, the method comprising (a) obtaining a detection signal for one or more probes on an array; (b) conducting one or more probe-specific analyses on the detection signal; and (c) determining a count of one or more nucleic acids based on results from the one or more probe-specific analyses.

Further disclosed herein are methods of determining a count of one or more molecules, the method comprising (a) hybridizing a plurality of molecules to a plurality of probes on an solid support to produce one or more hybridized molecules; and (b) determining a count of the one or more hybridized molecules by conducting one or more probe-specific analyses on the one or more probes.

Further disclosed herein are methods of determining a count of one or more molecules comprising (a) hybridizing a plurality of molecules to one or more probes on an array to produce one or more hybridized molecules; (b) counting individual probes on the array; (c) classifying the individual probes on the array based on the detection of the individual probes; and (d) determining a count of one or more molecules based on the classification of the individual probes.

Further disclosed herein are methods of determining a count of one or more molecules comprising (a) hybridizing a plurality of molecules to one or more probes on an array to produce one or more hybridized molecules; and (b) counting individual probes on the array, thereby determining a count of one or more molecules, wherein the method does not comprise labeling the plurality of molecules with barcodes, wherein the barcodes are used to differentiate two or more molecules of the same molecular species.

Further disclosed herein are methods of prenatal diagnostics. Generally, the method comprises (a) hybridizing a plurality of molecules from a sample from pregnant subject to one or more probes on a solid support; (b) conducting one or more probe-specific analyses of the one or more probes on the solid support; and (c) diagnosing, predicting, or monitoring a status or outcome of a fetal condition of a fetus in the pregnant subject based on results of the one or more probe-specific analyses.

Further disclosed herein are methods of prenatal diagnostics, the method comprising (a) hybridizing a plurality of nucleic acids from a sample from a pregnant subject to one or more probes on an array to produce one or more hybridized nucleic acids, wherein the plurality of nucleic acids may comprise one or more molecular species; (b) scanning the array to obtain a detection signal for the one or more probes; (c) converting the detection signal to a digital signal; (d) determining a count of the one or more hybridized nucleic acids based on a count of the digital signal; and (e) diagnosing, predicting, or monitoring a status or outcome of a fetal condition of a fetus in the pregnant subject based on the count of the one or more hybridized nucleic acids.

Further disclosed herein are methods of prenatal diagnostics, the method comprising (a) hybridizing a plurality of nucleic acids to one or more probes on an array to produce one or more hybridized nucleic acids; (b) detecting the one or more probes on the array to obtain a detection signal of the one or more probes; (c) determining a count of the one or more hybridized nucleic acids by applying an algorithm to the detection signal; and (d) diagnosing, predicting, or monitoring a status or outcome of a fetal condition of a fetus in the pregnant subject based on the count of the one or more hybridized nucleic acids.

Further disclosed herein are kits for array-based quantification. Generally, the kits for use in array-based quantification comprise a software program comprising computer-executable instructions for converting a detection signal from one or more probes to a digital signal. The kit for use in array-based quantification may comprise a software program comprising computer-executable instructions for conducting one or more probe-specific analyses of one or more probes on an array. The kit for use in array-based quantification may comprise a software program comprising computer-executable instructions for modeling probe behavior based on one or more probe features. The kit for use in array-based quantification may comprise a software program comprising computer-executable instructions for modeling probe behavior based on probe sequence. The kit for use in array-based quantification may comprise a software program comprising computer-executable instructions for modeling probe behavior using multivariate linear regression. The kits may further comprise a solid support comprising one or more probes.

Further disclosed herein are systems for array-based quantification. Generally, the systems comprise (a) a solid support comprising one or more probes; and (b) a computer readable medium comprising instructions for converting a detection signal from one or more probes to a digital signal.

The computer-implemented system may comprise (a) a digital processing device comprising an operating system configured to perform executable instructions and a memory device; and (b) a computer program including instructions executable by the digital processing device to determine a count of one or more nucleic acids hybridized to an array comprising (i) a software module configured to receive data input pertaining to one or more probes on the array; (ii) a software module configured to conduct probe-specific analysis of the data input and to generate an output based on the probe-specific analysis; and (iii) a software module configured to generate an output of the count of the one or more nucleic acid molecules hybridized to the array.

Array-Based Counting

Disclosed herein are methods, kits, and systems for array-based counting of one or more nucleic acids. The nucleic acids may be hybridized to one or more probes on an array. Determining the count of the one or more hybridized nucleic acids may comprise determining an intensity value for the probes. Alternatively, determining the count of the one or more nucleic acids may comprise obtaining a detection signal for one or more probes on an array. The detection signal may be an intensity value. The detection signal for the probe may be based on an image of the probe. The detection signal may be based on an image of the array. The image of the probe may be obtained by using a detection system. The detection system may comprise a microscope. The detection system may comprise a macroscope. The detection system may comprise a fluorescence detection system. The detection system may comprise a scanner. The scanner may be a flatbed scanner. The detection system may comprise a focus imaging system. The detection system may comprise a light source. The detection system may comprise a camera. The detection system may comprise an array reader.

Determining the count of the one or more hybridized nucleic acids may further comprise classifying the probes as positive or negative. The probes may be classified as positive if the intensity value of the probes is greater than a background intensity value. The background intensity value may be a probe-specific background intensity value. In some instances, the background intensity value is not common for all of the probes on the array. The background intensity value may be different for different probes. The background intensity value may be different for different groups of probes. An array may have multiple background intensity values depending on the probe. The probes may be classified as negative if the intensity value of the probes is less than a background intensity value. Determining the count of the one or more hybridized nucleic acids may further comprise determining a count of probes that may be classified as positive. The background intensity value may be referred to as a threshold.

Determining the count of the one or more hybridized nucleic acids may comprise use of a binary classification system. The binary classification system may be used to classify one or more probes on the solid support. Binary classification of the one or more probes may be based on signal from the probe. The signal may be an analogue signal. The binary classification system may convert an analogue signal of the probe to a digital signal.

Determining the count of one or more molecular species may comprise counting a plurality of hybridized nucleic acids. The plurality of hybridized nucleic acids may be counted concurrently. The plurality of hybridized nucleic acids may be counted sequentially. Counting may be based probe-specific analyses. Determining the count of one or more nucleic acids may comprise conducting probe-specific analyses of the detection signal of one or more probes.

The probe-specific analysis may comprise modeling a behavior of a probe based on a sequence of the probe. Modeling the behavior of the probe may comprise analysing one or more features of a probe and determining the effect of the feature on the detection signal of the probe. The one or more features may comprise nucleotide sequence, GC content, probe length, arrangement of the probe on the array. Probes with a high GC content may have a higher detection signal than probes with lower GC content. As such, the background signal from a probe with a high GC content may be greater than the background signal from a probe with a low GC content. Generally, probe-specific analysis may evaluate the effect of probe features on the detection signal and may result in different criteria for the analysis of the different probes and their detection signals. For example, a probe with a high background signal may require a higher detection signal than a probe with a low background signal to be counted.

The probe-specific analysis may comprise determining probe-specific background data. The probe-specific background data may be used to adjust the detection signal of a probe. The adjusted detection signal of the probe may be used to determine whether the probe is counted.

The probe-specific analysis may comprise clustering the probes on the array into groups based on similarity of one or more features. The one or more features may comprise GC content. The one or more features may comprise nucleotide sequence. The one or more features may comprise sequence homology. The one or more features may comprise probe length. The one or more features may comprise repetitive sequences. The one or more features may comprise arrangement of the probes on the array. The probe-specific analysis may comprise determining background values for specific probes or groups of probes. The probe-specific analysis may comprise adjusting a detection signal of a probe by subtracting a probe-specific background from the detection signal to produce an adjusted signal.

The probe-specific analysis may comprise calculating a t score for each probe.

The probe-specific analysis may comprise grouping the probes into one or more regions and calculating a mean trimmed t score for a region. The mean trimmed t score for a region may be calculated by (a) sorting the t score of the probes within the region; (b) removing the upper and lower 10% of the t scores; and (c) calculating the mean t score of the remaining t scores to produce the mean trimmed t score for the region. The mean trimmed t score may be calculated for regions comprising greater than 6 probes. The mean trimmed t score may be calculated for regions comprising 7, 8, 9, 10, 11, 12 or more probes. The mean trimmed t score may be calculated for regions comprising at least 7 probes. A region may have an average of 7 to 15 probes. A region may have an average of 10 to 11 probes.

The probe-specific analysis may comprise calculating a model-based analysis of tiling score (MATscore) for a probe. The MATscore may be calculated by multiplying a square root of the number of probes within a region by the trimmed mean t score of the region. The probe-specific analysis may comprise calculating a corrected MATscore. The corrected MATscore may be calculated by substracting a median MATscore from the MATscore. The median MATscore may be calculated by calculating the median of MATscores for a probe across two or more samples or arrays.

The method may comprise determining the count of one or more molecular species. The method may comprise determining the count of 1, 2, 3, 4, 5, 6, 7, 8, 9, 10, 11, 12, 13, 14, 15, 16, 17, 18, 19, 20 or more molecular species. The method may comprise determining the count of 25, 30, 35, 40, 45, 50, 55, 60, 65, 70, 75, 80, 85, 90, 95, 100 or more molecular species. The method may comprise determining the count of 100, 200, 300, 400, 500, 600, 700, 800, 900, 1000, 1100, 1200, 1300, 1400, 1500, 1600, 1700, 1800, 1900, 2000 or more molecular species. The method may comprise determining the count of 1000, 2000, 3000, 4000, 5000, 6000, 7000, 8000, 9000, 10000, 11000, 12000, 13000, 14000, 15000, 16000, 17000, 18000, 19000, 20000 or more molecular species. The method may comprise determining the count of 10000, 20000, 30000, 40000, 50000, 60000, 70000, 80000, 90000, 100000, 110000, 120000, 130000, 140000, 150000, 160000, 170000, 180000, 190000, 200000 or more molecular species. The method may comprise determining the count of two or more molecular species. The method may comprise determining the count of three or more molecular species. The method may comprise determining the count of four or more molecular species. The method may comprise determining the count of five or more molecular species. The method may comprise determining the count of six or more molecular species. The method may comprise determining the count of seven or more molecular species. The method may comprise determining the count of eight or more molecular species. The method may comprise determining the count of nine or more molecular species. The method may comprise determining the count of ten or more molecular species.

The one or more molecular species may comprise a genomic region or portion thereof. The one or more molecular species may comprise a chromosome or portion thereof. The chromosome may be chromosome 21. The chromosome may be chromosome 22. The one or more molecular species may comprise a gene or portion thereof. The one or more molecular species may comprise an exon or portion thereof. The one or more molecular species may comprise an intron or portion thereof. The one or more molecular species may comprise an untranslated region or portion thereof. The one or more molecular species may comprise a protein coding region or portion thereof. The one or more molecular species may comprise a non-coding region or portion thereof. The one or more molecular species may comprise a breakpoint junction or portion thereof. The one or more molecular species may comprise a structural variant or portion thereof.

The method disclosed herein may comprise determining a copy number of a first molecular species of the one or more molecular species. Determining the copy number of the first molecular species may comprise determining the relative copy number of the first molecular species. Determining the relative copy number of the first molecular species may comprise comparing a count of the first molecular species to a count of a second molecular species of the one or more molecular species. Determining the copy number of the first molecular species may comprise determining the absolute copy number of the first molecular species.

The methods disclosed herein may comprise shearing one or more nucleic acids of the plurality of nucleic acids to produce one or more sheared nucleic acids. The one or more nucleic acids may be sheared prior to hybridization of the plurality of nucleic acids to the one or more probes on the solid support. Shearing the one or more nucleic acids may comprise mechanical shearing. Shearing the one or more nucleic acids may comprise sonicating. Shearing the one or more nucleic acids may comprise use of one or more restriction enzymes.

The methods disclosed herein may comprise end polishing the one or more fragmented nucleic acids to produce one or more end polished nucleic acids.

The methods disclosed herein may comprise polyA tailing the one or more polished nucleic acids to produce one or more polyA tailed nucleic acids.

The methods disclosed herein may comprise ligating one or more adaptors to the one or more polyA tailed nucleic acids to produce one or more adaptor ligated nucleic acids. The adaptors may comprise a barcode, universal primer sequence, sequencing primer, or a combination thereof.

The methods disclosed herein may comprise estimating a concentration of the one or more adaptor ligated nucleic acids. Estimating the concentration of the one or more adaptor ligated nucleic acids may comprise conducting a quantitative PCR reaction.

The methods disclosed herein may comprise conducting an amplification reaction on the one or more adaptor ligated nucleic acids to produce one or more amplified nucleic acids. The methods disclosed herein may comprise conducting an amplification reaction on the plurality of nucleic acids to produce one or more amplified nucleic acids, wherein the plurality of nucleic acids may comprise one or more adaptor ligated nucleic acids or non-adaptor ligated nucleic acids.

The methods disclosed herein may comprise fragmenting the one or more amplified nucleic acids to produce one or more fragmented nucleic acids.

The methods disclosed herein may comprise labeling the one or more fragmented nucleic acids to produce one or more labeled nucleic acids. Labeling the one or more fragmented nucleic acids may comprise DNA end labeling. Labeling the one or more fragmented nucleic acids may comprise labeling with a terminal transferase. Labeling the one or more fragmented nucleic acids may comprise labeling with a biotinylated nucleotide.

The methods disclosed herein may comprise hybridizing the plurality of nucleic acids to the one or more probes on the solid support. The methods disclosed herein may comprise hybridizing the one or more labeled nucleic acids to the one or more probes on the solid support.

The methods disclosed herein may comprise staining the one or more hybridized nucleic acids.

The methods disclosed herein may comprise scanning the solid support to detect to the one or more hybridized nucleic acids.

Solid Supports

The methods, kits, and systems disclosed herein may comprise one or more solid supports or uses thereof. The solid support may be an array. The kit may comprise 1, 2, 3, 4, 5, 6, 7, 8, 9, 10, 11, 12, 13, 14, 15, 16, 17, 18, 19, 20 or more arrays. The kit may comprise 2 or more arrays. The kit may comprise 3 or more arrays. The kit may comprise 4 or more arrays. The kit may comprise 5 or more arrays. The kit may comprise 6 or more arrays.

Examples of arrays include, but are not limited to, DNA microarrays, MMChips, protein microarrays, peptide microarrays, tissue microarrays, cellular microarrays (e.g., transfection microarrays), chemical compound microarrays, antibody microarrays, carbohydrate arrays (e.g., glycoarrays), phenotype microarrays, reverse Phase Protein Microarrays, microarrays of lysates or serum, and interferometric reflectance imaging sensor (IRIS). The array may be a DNA microarray. The DNA microarray may be a cDNA microarray, oligonucleotide microarray, BAC microarray and SNP microarray. The array may be a microarray. The array may be a solid-phase microarray. The array may be a bead-based array.

The array may be a tiling array. The tiling array may comprise overlapping probes (e.g., tiling probes) designed to densely represent a genomic region of interest. The genomic region of interest may comprise an entire chromosome. The genomic region of interest may comprise at least a portion of a chromosome. The genomic region of interest may comprise at least a portion of two or more chromosomes. The genomic region of interest may comprise chromosome 21, 22, or both. The genomic region of interest may comprise chromosome 13, 15, 16, 18, 21, 22, X, Y, or a combination thereof. The genomic region of interest may comprise a gene locus. The genomic region of interest may comprise a protein-coding region. A protein-coding region may comprise an exon, intron, untranslated region, or a combination thereof. The genomic region of interest may comprise a non-coding region. Generally, a non-coding region may refer to a portion of the genome that does not encode for a protein. Examples of non-coding regions include, but are not limited to, rRNA, tRNA, miRNA, siRNA, snoRNA, and lncRNA.

The array may comprise a plurality of probes. The plurality of probes may comprise 10 or more probes. The plurality of probes may comprise 20 or more probes. The plurality of probes may comprise 30, 40, 50, 60, 70, 80, 90, 100 or more probes. The plurality of probes may comprise 125, 150, 175, 200, 225, 250, 275, 300, 325, 350, 375, 400, 425, 450, 475, 500 or more probes. The plurality of probes may comprise 550, 600, 650, 700, 750, 800, 850, 900, 950, 1000 or more probes. The plurality of probes may comprise 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000 or more probes. The plurality of probes may comprise 20000, 30000, 40000, 50000, 60000, 70000, 80000, 90000, 100000 or more probes. The plurality of probes may comprise 125000, 150000, 175000, 200000, 225000, 250000, 275000, 300000 or more probes. The plurality of probes may comprise 40000 or more probes. The plurality of probes may comprise 45000 or more probes. The plurality of probes may comprise 50000 or more probes. The plurality of probes may comprise 60000 or more probes. The plurality of probes may comprise 110000 or more probes.

The plurality of probes may comprise two or more identical probes. At least about 1% of the probes of the plurality of probes may be identical. At least about 5% of the probes of the plurality of probes may be identical. At least about 10% of the probes of the plurality of probes may be identical. At least about 15% of the probes of the plurality of probes may be identical. At least about 20% of the probes of the plurality of probes may be identical. At least about 25%, 30%, 35%, 40%, 45%, or 50% of the probes of the plurality of probes may be identical.

The plurality of probes may comprise two or more different probes. At least about 1%, 5%, 10%, 15%, 20%, 25%, 30%, 35%, 40%, 45%, or 50% of the probes of the plurality of probes may be different. At least about 40% of the probes of the plurality of probes may be different. At least about 50% of the probes of the plurality of probes may be different. At least about 10% of the probes of the plurality of probes may be different. At least about 60% of the probes of the plurality of probes may be different. At least about 70% of the probes of the plurality of probes may be different. At least about 75% of the probes of the plurality of probes may be different. At least about 80% of the probes of the plurality of probes may be different. At least about 85% of the probes of the plurality of probes may be different. At least about 90% of the probes of the plurality of probes may be different. At least about 95% of the probes of the plurality of probes may be different.

Probes

The methods, kits, and systems disclosed herein may comprise one or more probes or uses thereof. The probes may be attached to one or more solid supports. The probes may be used to digitally quantify one or more molecules.

The methods, kits, and systems may comprise 1, 2, 3, 4, 5, 6, 7, 8, 9, 10, 11, 12, 13, 14, 15, 16, 17, 18, 19, 20 or more probes or uses thereof. The methods, kits, and systems may comprise 30, 40, 50, 60, 70, 80, 90, 100, 110, 120, 130, 140, 150, 160, 170, 180, 190, 200 or more probes or uses thereof. The methods, kits, and systems may comprise 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000 or more probes or uses thereof. The methods, kits, and systems may comprise 10000, 20000, 30000, 40000, 50000, 60000, 70000, 80000, 90000, 100000, 110000, 120000, 130000, 140000, 150000, 160000, 170000, 180000, 190000, 200000 or more probes or uses thereof. The methods, kits, and systems may comprise 100000, 200000, 300000, 400000, 500000, 600000, 700000, 800000, 900000, 1000000, 1100000, 1200000, 1300000, 1400000, 1500000, 1600000, 1700000, 1800000, 1900000, 2000000 or more probes or uses thereof. The methods, kits, and systems may comprise 1000000, 2000000, 3000000, 4000000, 5000000, 6000000, 7000000, 8000000, 9000000, 10000000, 11000000, 12000000, 13000000, 14000000, 15000000, 16000000, 17000000, 18000000, 19000000, 20000000 or more probes or uses thereof.

The methods, kits, and systems disclosed herein may comprise one or more sets of probes, or uses thereof. The methods, kits, and systems disclosed herein may comprise 2, 3, 4, 5, 6, 7, 8, 9, 10, 11, 12, 13, 14, 15, 16, 17, 18, 19, 20 or more sets of probes, or uses thereof. Probes within a set of probes may be different. Probes within a set of probes may be the same. Probes within a set of probes may contain regions of similarity. Probes within a set of probes may contain one or more identical regions. Probes within a set of probes may contain one or more different regions. Probes from two or more sets of probes may be different. Probes from two or more sets may be the same. Probes from two or more sets may contain regions of similarity. Probes from two or more sets may contain one or more identical regions. Probes from two or more sets may contain one or more different regions. For example, a kit may comprise at least two sets of probes.

A probe may comprise a plurality of nucleotides. Two or more probes may be partially identical. The two or more probes may contain 3, 4, 5, 6, 7, 8, 9, 10, 11, 12, 13, 14, 15, 16, 17, 18, 19, 20 or more identical nucleotides. The two or more probes may contain 3 or more identical nucleotides. The two or more probes may contain 5 or more identical nucleotides. The two or more probes may contain 7 or more identical nucleotides. The two or more probes may contain 9 or more identical nucleotides. The two or more probes may contain 12 or more identical nucleotides. The two or more probes may contain 15 or more identical nucleotides. Two or more probes may contain 20 or more identical nucleotides. The nucleotides may be consecutive. The nucleotides may be non-consecutive.

Two or more probes may differ by 1, 2, 3, 4, 5, 6, 7, 8, 9, 10, 11, 12, 13, 14, 15, 16, 17, 18, 19, 20 or more nucleotides. Two or more probes may differ by one or more nucleotides. Two or more probes may differ by two or more nucleotides. Two or more probes may differ by three or more nucleotides. Two or more probes may differ by four or more nucleotides. Two or more probes may differ by five or more nucleotides. Two or more probes may differ by six or more nucleotides. Two or more probes may differ by seven or more nucleotides. Two or more probes may differ by eight or more nucleotides. Two or more probes may differ by ten or more nucleotides. Two or more probes may differ by 12 or more nucleotides. Two or more probes may differ by 15 or more nucleotides. Two or more probes may differ by 20 or more nucleotides.

The plurality of probes may comprise two or more tiling array probes. The terms tiling array probes, tiling probes and overlapping probes may be used interchangeably. The tiling array probes may be overlapping probes. Tiling array probes may target contiguous regions of a genome. At least about 5%, 6%, 7%, 8%, 9%. 10%, 11%, 12%, 13%, 14%, 15%, 20%, 25%, 30%, 35%, 40%, 45%, 50%, 55%, 60%, 65%, 70%, 75%, 80%, 85%, 90%, or 95% probes in the plurality of probes may be tiling array probes. At least about 5% of probes in the plurality of probes may be tiling probes. At least about 10% of probes in the plurality of probes may be tiling array probes. At least about 15% of probes in the plurality of probes may be tiling array probes. At least about 25% of probes in the plurality of probes may be tiling array probes. At least about 50% of probes in the plurality of probes may be tiling probes. At least about 75% of probes in the plurality of probes may be tiling probes. At least about 80% of probes in the plurality of probes may be tiling probes.

The probes on the solid support may hybridize to one or more molecules. The probes on the solid support may hybridize to two or more molecules. The probes on the solid support may hybridize to 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100 or more molecules. The probes on the solid support may hybridize to 200, 300, 400, 500, 600, 700, 800, 900, 1000 or more molecules. The probes on the solid support may hybridize to 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000 or more molecules. The molecules may be of the same molecular species. The molecules may be of different molecular species.

The probes may bind to one or more regions on a molecule. The probes may bind to 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more regions on a molecule. The probes may bind to two or more regions on a molecules. The probes may bind to three or more regions on a molecule. The probes may bind to four or more regions on a molecule. The probes may bind to five or more regions on a molecule. The probes may bind to ten or more regions on a molecule.

The probes may bind to two or more regions on a molecule. The two or more regions may overlap. The two or more regions on the molecule may overlap by at least about 1, 2, 3, 4, 5, 6, 7, 8, 9, 10, 11, 12, 13, 14, 15, 16, 17, 18, 19, 20 or more nucleotides. The two or more regions on the molecule may overlap by at least about 25, 30, 35, 40, 45, 50, 55, 60, 65, 70, 75, 80, 85, 90, 95, 100 or more nucleotides. The two or more regions on the molecule may overlap by at least about 1 nucleotide. The two or more regions on the molecule may overlap by at least about 2 nucleotides. The two or more regions on the molecules may overlap by at least about 3 nucleotides. The two or more regions on the molecules may overlap by at least about 4 nucleotides. The two or more regions on the molecules may overlap by at least about 5 nucleotides. The two or more regions on the molecules may overlap by at least about 7 nucleotides. The two or more regions on the molecules may overlap by at least about 10 nucleotides. The two or more regions on the molecules may overlap by at least about 15 nucleotides. The two or more regions on the molecules may overlap by at least about 20 nucleotides.

Labels

The methods, kits, and systems disclosed herein may comprise one or more labels or uses thereof. The methods, kits, and systems may comprise 1, 2, 3, 4, 5, 6, 7, 8, 9, 10, 11, 12, 13, 14, 15, 16, 17, 18, 19, 20 or more labels or uses thereof. The methods, kits, and systems may comprise 30, 40, 50, 60, 70, 80, 90, 100, 110, 120, 130, 140, 150, 160, 170, 180, 190, 200 or more labels or uses thereof. The methods, kits, and systems may comprise 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000 or more labels or uses thereof. The methods, kits, and systems may comprise 10000, 20000, 30000, 40000, 50000, 60000, 70000, 80000, 90000, 100000, 110000, 120000, 130000, 140000, 150000, 160000, 170000, 180000, 190000, 200000 or more labels or uses thereof. The methods, kits, and systems may comprise 100000, 200000, 300000, 400000, 500000, 600000, 700000, 800000, 900000, 1000000, 1100000, 1200000, 1300000, 1400000, 1500000, 1600000, 1700000, 1800000, 1900000, 2000000 or more labels or uses thereof. The methods, kits, and systems may comprise 1000000, 2000000, 3000000, 4000000, 5000000, 6000000, 7000000, 8000000, 9000000, 10000000, 11000000, 12000000, 13000000, 14000000, 15000000, 16000000, 17000000, 18000000, 19000000, 20000000 or more labels or uses thereof.

The methods, kits, and systems disclosed herein may comprise one or more sets of labels, or uses thereof. The methods, kits, and systems disclosed herein may comprise 2, 3, 4, 5, 6, 7, 8, 9, 10, 11, 12, 13, 14, 15, 16, 17, 18, 19, 20 or more sets of labels, or uses thereof. Labels within a set of labels may be different. Labels within a set of labels may be the same. Labels within a set of labels may contain regions of similarity. Labels within a set of labels may contain one or more identical regions. Labels within a set of labels may contain one or more different regions. Labels from two or more sets of labels may be different. Labels from two or more sets may be the same. Labels from two or more sets may contain regions of similarity. Labels from two or more sets may contain one or more identical regions. Labels from two or more sets may contain one or more different regions. For example, a kit may comprise at least two sets of labels

Adaptors

The methods, kits, and systems disclosed herein may comprise one or more adaptors or uses thereof. Adaptors may enable attachment of a probe to a molecule or product thereof. Adaptors may enable attachment of a label to a molecule or product thereof. Adaptors may enable attachment of a detectable label to a molecule or product thereof. Products of a molecule may include, but are not limited to, molecules that have been sheared, fragmented, enriched, purified, isolated, amplified, labeled, ligated, captured, hybridized, polyA-tailed, end-polished, or a combination thereof. Adaptors may enable attachment of a probe to a labeled-molecule. Adaptors may enable attachment of a probe to a label. Adaptors may enable attachment of a probe to a detectable label. Adaptors may enable attachment of a label to a detectable label.

The methods, kits, and systems may comprise 1, 2, 3, 4, 5, 6, 7, 8, 9, 10, 11, 12, 13, 14, 15, 16, 17, 18, 19, 20 or more adaptors or uses thereof. The methods, kits, and systems may comprise 30, 40, 50, 60, 70, 80, 90, 100, 110, 120, 130, 140, 150, 160, 170, 180, 190, 200 or more adaptors or uses thereof. The methods, kits, and systems may comprise 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000 or more adaptors or uses thereof. The methods, kits, and systems may comprise 10000, 20000, 30000, 40000, 50000, 60000, 70000, 80000, 90000, 100000, 110000, 120000, 130000, 140000, 150000, 160000, 170000, 180000, 190000, 200000 or more adaptors or uses thereof. The methods, kits, and systems may comprise 100000, 200000, 300000, 400000, 500000, 600000, 700000, 800000, 900000, 1000000, 1100000, 1200000, 1300000, 1400000, 1500000, 1600000, 1700000, 1800000, 1900000, 2000000 or more adaptors or uses thereof. The methods, kits, and systems may comprise 1000000, 2000000, 3000000, 4000000, 5000000, 6000000, 7000000, 8000000, 9000000, 10000000, 11000000, 12000000, 13000000, 14000000, 15000000, 16000000, 17000000, 18000000, 19000000, 20000000 or more adaptors or uses thereof.

The methods, kits, and systems disclosed herein may comprise one or more sets of adaptors, or uses thereof. The methods, kits, and systems disclosed herein may comprise 2, 3, 4, 5, 6, 7, 8, 9, 10, 11, 12, 13, 14, 15, 16, 17, 18, 19, 20 or more sets of adaptors, or uses thereof. Adaptors within a set of adaptors may be different. Adaptors within a set of adaptors may be the same. Adaptors within a set of adaptors may contain regions of similarity. Adaptors within a set of adaptors may contain one or more identical regions. Adaptors within a set of adaptors may contain one or more different regions. Adaptors from two or more sets of adaptors may be different. Adaptors from two or more sets may be the same. Adaptors from two or more sets may contain regions of similarity. Adaptors from two or more sets may contain one or more identical regions. Adaptors from two or more sets may contain one or more different regions. For example, a kit may comprise at least two sets of adaptors.

Nucleic Acids

The methods, kits, and systems disclosed herein may be used to quantify one or more molecules hybridized to one or more probes on a solid support. The molecules may be nucleic acids. The nucleic acids may be DNA. The DNA may be a cDNA. The nucleic acids may be RNA. The RNA may be mRNA. The RNA may be cRNA (e.g., antisense RNA). The RNA may be sense RNA. The nucleic acid may be double-stranded. The nucleic acid may be single-stranded. The nucleic acids may be cell-free nucleic acids. The nucleic acids may be circulating nucleic acids.

The method, kits, and systems disclosed herein may be used to determine a count of one or more nucleic acids from a sample. A total quantity of nucleic acids in the sample may be less than 1 genome equivalent. A total quantity of nucleic acids in the sample may be less than 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 genome equivalent. A total quantity of nucleic acids in the sample may be less than 0.7 genome equivalent. A total quantity of nucleic acids in the sample may be less than 0.5 genome equivalent. A total quantity of nucleic acids in the sample may be less than 0.3 genome equivalent. A total quantity of nucleic acids in the sample may be less than 0.1 genome equivalent. A total quantity of nucleic acids in the sample may be less than 1 haploid genome equivalent. A total quantity of nucleic acids in the sample may be less than 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001 haploid genome equivalent. A total quantity of nucleic acids in the sample may be less than 0.9 haploid genome equivalent. A total quantity of nucleic acids in the sample may be less than 0.7 haploid genome equivalent. A total quantity of nucleic acids in the sample may be less than 0.5 haploid genome equivalent. A total quantity of nucleic acids in the sample may be less than 0.3 haploid genome equivalent. A total quantity of nucleic acids in the sample may be less than 0.1 haploid genome equivalent. A total quantity of nucleic acids in the sample may be less than 0.05 haploid genome equivalent. A total quantity of nucleic acids in the sample may be less than 0.01 haploid genome equivalent.

A nucleic acid or product thereof may be less than about 2000, 1900, 1800, 1700, 1600, 1500, 1400, 1300, 1200, 1100, 1000, 900, 800, 700, 600, or 500 basepairs in length. A nucleic acid or product thereof may be less than about 500 basepairs in length. A nucleic acid or product thereof may be less than about 400 basepairs in length. A nucleic acid or product thereof may be less than about 300 basepairs in length. A nucleic acid or product thereof may be less than about 200 basepairs in length. A nucleic acid or product thereof may be less than about 190 basepairs in length. A nucleic acid or product thereof may be less than about 180 basepairs in length. A nucleic acid or product thereof may be less than about 170 basepairs in length. A nucleic acid or product thereof may be less than about 160 basepairs in length. A nucleic acid or product thereof may be less than about 100 basepairs in length. A nucleic acid or product thereof may be less than about 90 basepairs in length.

A nucleic acid or product thereof may be greater than about 10, 20, 30, 40, 50, 60, 70, 80, 90, 100, 120, 140, 160, 180, 200, 250, or 300 basepairs in length. A nucleic acid or product thereof may be greater than about 10 basepairs in length. A nucleic acid or product thereof may be greater than about 20 basepairs in length. A nucleic acid or product thereof may be greater than about 30 basepairs in length. A nucleic acid or product thereof may be greater than about 40 basepairs in length. A nucleic acid or product thereof may be greater than about 50 basepairs in length. A nucleic acid or product thereof may be greater than about 60 basepairs in length. A nucleic acid or product thereof may be greater than about 70 basepairs in length.

A nucleic acid or product thereof may be between about 10 to about 1000 basepairs in length. A nucleic acid or product thereof may be between about 10 to about 800 basepairs in length. A nucleic acid or product thereof may be between about 10 to about 600 basepairs in length. A nucleic acid or product thereof may be between about 10 to about 500 basepairs in length. A nucleic acid or product thereof may be between about 10 to about 400 basepairs in length. A nucleic acid or product thereof may be between about 10 to about 200 basepairs in length. A nucleic acid or product thereof may be between about 30 to about 500 basepairs in length. A nucleic acid or product thereof may be between about 30 to about 400 basepairs in length. A nucleic acid or product thereof may be between about 30 to about 300 basepairs in length. A nucleic acid or product thereof may be between about 30 to about 200 basepairs in length. A nucleic acid or product thereof may be between about 40 to about 200 basepairs in length. A nucleic acid or product thereof may be between about 40 to about 170 basepairs in length.

The nucleic acids may be from a sample. The sample may be blood, plasma, a blood fraction, saliva, sputum, urine, semen, transvaginal fluid, cerebrospinal fluid, stool, a cell or a tissue biopsy. The sample may be blood or plasma. The sample may be urine.

The sample may be from a subject. The subject may be a human. The subject may be a mammal. The subject may be a non-human primate (e.g., apes, monkeys, chimpanzees), cat, dog, rabbit, goat, horse, cow, pig, and sheep. The subject may be male or female. The subject may be a fetus, infant, child, adolescent, teenager or adult. The subject may be a non-mammal. Non-mammals include, but are not limited to, reptiles, amphibians, avians, and fish. A reptile may be a lizard, snake, alligator, turtle, crocodile, and tortoise. An amphibian may be a toad, frog, newt, and salamander. Examples of avians include, but are not limited to, ducks, geese, penguins, ostriches, and owls. Examples of fish include, but are not limited to, catfish, eels, sharks, and swordfish.

Kits

Disclosed herein are solid support-based digital quantification kits. The kits may be used to determine a quantity of one or more molecules. The kits may be used to determine an absolute abundance of one or more molecules. The kits may be used to determine a relative abundance of one or more molecules. The kits may be used to determine an abundance of one or more molecules from one or more samples. The kits may be used to determine an abundance of one or more molecules from two or more samples. The two or more samples may be from a single subject. Alternatively, the two or more samples are from two or more different subjects. The two or more samples may be obtained at the same time point. The two or more samples may be taken at two or more different time points.

A kit for array-based counting may comprise a software program comprising computer-executable instructions for counting one or more objects hybridized to one or more probes on an array. The software program may comprise computer-executable instructions for scanning the array. The software program may comprise computer-executable instructions for detecting the one or more objects hybridized to the one or more probes on the array. The software program may comprise computer-executable instructions for measuring a detection signal emitted from the one or more objects or probes. The software program may comprise computer-executable instructions for converting the detection signal to a digital signal. The software program may comprise computer-executable instructions for measuring an analogue signal emitted from the one or more objects or probes. The software program may comprise computer-executable instructions for converting the analogue signal to a digital signal. The software program may comprise computer-executable instructions for classifying the one or more probes on the array. The kit may comprise one or more arrays.

The kit may comprise one or more labels. The kit may comprise a plurality of labels. The kit may comprise one or more sets of labels. The kit may comprise a first container comprising a first plurality of labels and a second container comprising a second plurality of labels. Labels from the first set of labels may differ from labels from the second set of labels by one or more features.

The kit may comprise one or more containers comprising a plurality of labels. The kit may comprise instructions for attaching the plurality of labels to one or more molecules from a sample.

The kit may comprise one or more control nucleic acids. The kit may comprise a detection system.

Digital Processing Device

The digital array-based quantification methods, kits, and systems described herein may include a digital processing device, or use of the same. The digital processing device may include one or more hardware central processing units (CPU) that carry out the device's functions. The digital processing device may further comprise an operating system configured to perform executable instructions. The digital processing device may optionally be connected a computer network. The digital processing device may optionally be connected to the Internet such that it accesses the World Wide Web. The digital processing device may optionally be connected to a cloud computing infrastructure. The digital processing device may optionally be connected to an intranet. The digital processing device may optionally be connected to a data storage device.

In accordance with the description herein, suitable digital processing devices may include, by way of non-limiting examples, server computers, desktop computers, laptop computers, notebook computers, sub-notebook computers, netbook computers, netpad computers, set-top computers, handheld computers, internet appliances, mobile smartphones, tablet computers, personal digital assistants, video game consoles, and vehicles. Those of skill in the art will recognize that many smartphones may be suitable for use in the system described herein. Those of skill in the art will also recognize that select televisions, video players, and digital music players with optional computer network connectivity may be suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which manages the device's hardware and provides services for execution of applications. Those of skill in the art will recognize that suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems may include, by way of non-limiting examples, Microsoft® Windows®, Apple® Mac OS X®, UNIX®, and UNIX-like operating systems such as GNU/Linux®. The operating system may provided by cloud computing. Those of skill in the art will also recognize that suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

The device may include a storage and/or memory device. The storage and/or memory device may be one or more physical apparatuses used to store data or programs on a temporary or permanent basis. The device may be volatile memory and may require power to maintain stored information. The device may be non-volatile memory and may retain stored information when the digital processing device is not powered. The non-volatile memory may comprise flash memory. The non-volatile memory comprises dynamic random-access memory (DRAM). The non-volatile memory may comprise ferroelectric random access memory (FRAM). The non-volatile memory may comprise phase-change random access memory (PRAM). The device may be a storage device including, by way of non-limiting examples, CD-ROMs, DVDs, flash memory devices, magnetic disk drives, magnetic tapes drives, optical disk drives, and cloud computing based storage. The storage and/or memory device may be a combination of devices such as those disclosed herein.

The digital processing device may include a display to send visual information to a user. The display may be a cathode ray tube (CRT). The display may be a liquid crystal display (LCD). The display may be a thin film transistor liquid crystal display (TFT-LCD). The display may be an organic light emitting diode (OLED) display. An OLED display may be a passive-matrix OLED (PMOLED) or active-matrix OLED (AMOLED) display. The display may be a plasma display. The display may be a video projector. The display may be a combination of devices such as those disclosed herein.

The digital processing device may include an input device to receive information from a user. The input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera to capture motion or visual input. The input device may be a combination of devices such as those disclosed herein.

Non-Transitory Computer Readable Storage Medium

The digital array-based quantification methods, kits, and systems disclosed herein may include one or more non-transitory computer readable storage media encoded with a program including instructions executable by the operating system of an optionally networked digital processing device. A computer readable storage medium may be a tangible component of a digital processing device. A computer readable storage medium may be optionally removable from a digital processing device. A computer readable storage medium may include, by way of non-limiting examples, CD-ROMs, DVDs, flash memory devices, solid state memory, magnetic disk drives, magnetic tape drives, optical disk drives, cloud computing systems and services, and the like. The program and instructions may be permanently, substantially permanently, semi-permanently, or non-transitorily encoded on the media.

Computer Program

The digital array-based quantification methods, kits, and systems disclosed herein may include at least one computer program, or use of the same. A computer program may include a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. A computer program may comprise one sequence of instructions. A computer program comprises a plurality of sequences of instructions. A computer program may be provided from one location. A computer program may be provided from a plurality of locations. A computer program may include one or more software modules. A computer program may include, in part or in whole, one or more web applications, one or more mobile applications, one or more standalone applications, one or more web browser plug-ins, extensions, add-ins, or add-ons, or combinations thereof.

Web Application

A computer program may include a web application. In light of the disclosure provided herein, those of skill in the art will recognize that a web application, in various embodiments, utilizes one or more software frameworks and one or more database systems. A web application may be created upon a software framework such as Microsoft® .NET or Ruby on Rails (RoR). A web application may utilize one or more database systems including, by way of non-limiting examples, relational, non-relational, object oriented, associative, and XML database systems. Suitable relational database systems may include, by way of non-limiting examples, Microsoft® SQL Server, mySQL™, and Oracle®. Those of skill in the art will also recognize that a web application may be written in one or more versions of one or more languages. A web application may be written in one or more markup languages, presentation definition languages, client-side scripting languages, server-side coding languages, database query languages, or combinations thereof. A web application may be written to some extent in a markup language such as Hypertext Markup Language (HTML), Extensible Hypertext Markup Language (XHTML), or eXtensible Markup Language (XML). A web application may be written to some extent in a presentation definition language such as Cascading Style Sheets (CSS). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. A web application may be written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

Mobile Application

A computer program may include a mobile application provided to a mobile digital processing device. The mobile application may be provided to a mobile digital processing device at the time it may be manufactured. The mobile application may be provided to a mobile digital processing device via the computer network described herein.

In view of the disclosure provided herein, a mobile application may be created by techniques known to those of skill in the art using hardware, languages, and development environments known to the art. Those of skill in the art will recognize that mobile applications may be written in several languages. Suitable programming languages may include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments may be available from several sources. Commercially available development environments may include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers may distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums may be available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

A computer program may include a standalone application, which may be a program that may be run as an independent computer process, not an add-on to an existing process, e.g., not a plug-in. Those of skill in the art will recognize that standalone applications may often be compiled. A compiler may be a computer program(s) that transforms source code written in a programming language into binary object code such as assembly language or machine code. Suitable compiled programming languages may include, by way of non-limiting examples, C, C++, Objective-C, COBOL, Delphi, Eiffel, Java™, Lisp, Python™, Visual Basic, and VB .NET, or combinations thereof. Compilation may often be performed, at least in part, to create an executable program. A computer program may include one or more executable complied applications.

Web Browser Plug-in

The computer program may include a web browser plug-in. In computing, a plug-in may be one or more software components that add specific functionality to a larger software application. Makers of software applications support plug-ins to enable third-party developers to create abilities which extend an application, to support easily adding new features, and to reduce the size of an application. When supported, plug-ins may enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar comprises one or more web browser extensions, add-ins, or add-ons. The toolbar comprises one or more explorer bars, tool bands, or desk bands.

In view of the disclosure provided herein, those of skill in the art will recognize that several plug-in frameworks may be available that enable development of plug-ins in various programming languages, including, by way of non-limiting examples, C++, Delphi, Java™, PHP, Python™, and VB .NET, or combinations thereof.

Web browsers (also called Internet browsers) may be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser may be a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The digital array-based quantification methods, kits, and systems disclosed herein may include software, server, and/or database modules, or use of the same. In view of the disclosure provided herein, software modules may be created by techniques known to those of skill in the art using machines, software, and languages known to the art. The software modules disclosed herein may be implemented in a multitude of ways. A software module may comprise a file, a section of code, a programming object, a programming structure, or combinations thereof. In further various embodiments, a software module comprises a plurality of files, a plurality of sections of code, a plurality of programming objects, a plurality of programming structures, or combinations thereof. The one or more software modules may comprise, by way of non-limiting examples, a web application, a mobile application, and a standalone application. The software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

The software program may comprise computer-executable instructions for conducting one or more probe-specific analyses. The software program may comprise computer-executable instructions for modeling probe behavior. The software program may comprise computer-executable instructions for applying an algorithm to a detection signal of a probe. The software program may comprise computer-executable instructions for classifying a probe on the array. The software program may comprise computer-executable instructions for calculating a probe-specific background. The software program may comprise computer-executable instructions for calculating a t score of a probe. The software program may comprise computer-executable instructions for calculating a median trimmed t score of a region comprising a plurality of probes. The software program may comprise computer-executable instructions for calculating a MATscore. The software program may comprise computer-executable instructions for calculating a median MATscore. The software program may comprise computer-executable instructions for calculating a corrected MATscore. The software program may comprise computer-executable instructions for counting probes or objects on an array. The software program may comprise computer-executable instructions for scanning the array. The software program may comprise computer-executable instructions for detecting the one or more objects hybridized to the one or more probes on the array. The software program may comprise computer-executable instructions for detecting the one or more probes on the array. The software program may comprise computer-executable instructions for measuring an analogue signal emitted from the one or more objects. The software program may comprise computer-executable instructions for measuring an analogue signal emitted from the one or more probes. The software program may comprise computer-executable instructions for converting a detection signal to a digital signal. The software program may comprise computer-executable instructions for converting an analogue signal to a digital signal.

The analogue signal may be an intensity signal. The intensity signal may be a luminescence signal. Examples of luminescence signals include, but are not limited to, chemiluminescence, bioluminescence, electrochemiluminescence, photoluminescence, fluorescence, phosphorescence, and radioluminescence. The intensity signal may be fluorescence intensity.

The software program may comprise computer-executable instructions for converting the analogue signal to a digitized signal. Converting the analogue signal to a digitized signal may comprise substracting a background analogue signal from the analogue signal emitted from the one or more objects to produce a background adjusted analogue signal. Converting the analogue signal may comprise comparing the background adjusted analogue signal to a control analogue value. The analogue signal may be converted to a countable digitized signal if the background adjusted analogue signal may be greater than the control analogue value. The analogue signal may be converted to a non-countable digitized signal if the background adjusted analogue signal may be less than the control analogue value.

The instructions may comprise computer-executable instructions for counting a number of countable digital signals, thereby digitally counting the one or more objects.

Databases

The digital array-based quantification methods, kits, and systems disclosed herein may include one or more databases, or use of the same. In view of the disclosure provided herein, those of skill in the art will recognize that many databases may be suitable for storage and retrieval of bioinformatic information. Suitable databases include, by way of non-limiting examples, relational databases, non-relational databases, object oriented databases, object databases, entity-relationship model databases, associative databases, and XML databases. A database may be internet-based. A database may be web-based. A database may be cloud computing-based. A database may be based on one or more local computer storage devices.

Applications

The methods and kits disclosed herein may be used in diagnosing, prognosing and/or monitoring a status or outcome of a disease or condition in a subject in need thereof. The method may comprise (a) obtaining a sample from a subject; (b) hybridizing at least a portion of the sample from the subject to a solid support, wherein the portion of the sample has a concentration of less than 1 genome equivalent; (c) detecting one or more nucleic acids hybridized to the solid support; and (d) diagnosing, prognosing, and/or monitoring a status or outcome of a disease or condition based on the detection of the one or more nucleic acids. The method may further comprise determining a count of one or more nucleic acids based on the detection of the one or more nucleic acids. The method may further comprise determining a count of 100 or more nucleic acids based on the detection of the one or more nucleic acids. The method may further comprise determining a count of 10000 or more nucleic acids based on the detection of the one or more nucleic acids. The method may further comprise determining a count of 40000 or more nucleic acids based on the detection of the one or more nucleic acids. Detecting the one or more nucleic acids may comprise detecting individual nucleic acids hybridized to the solid support. Detecting the one or more nucleic acids may comprise detecting a plurality of nucleic acids. The plurality of nucleic acids may comprise one or more molecular species. The plurality of nucleic acids may comprise two or more molecular species. The plurality of nucleic acids may comprise 50000 or more nucleic acids. The solid support may comprise a plurality of probes. The one or more nucleic acids may hybridize to one or more probes. The plurality of probes may comprise 150 or more probes. The plurality of probes may comprise 300 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The sample may be a blood or plasma sample. The sample may be a urine sample. The sample may be a stool sample.

The method of diagnosing, prognosing and/or monitoring a status or outcome of a disease or condition in a subject may comprise (a) obtaining a sample from a subject, wherein the sample comprises a plurality of nucleic acids; (b) hybridizing the plurality of nucleic acids to one or more probes on a solid support to produce one or more hybridized nucleic acids, wherein the plurality of nucleic acids comprise one or more molecular species; and (c) determining a count of the one or more hybridized nucleic acids by counting individual probes on the solid support. Alternatively, the method comprises (a) hybridizing a plurality of nucleic acids to a plurality of probes on a solid support to produce one or more hybridized nucleic acids, wherein the plurality of probes comprises 150 or more different probes; and (b) determining a count of the one or more hybridized nucleic acids by counting individual probes on the solid support. The method may comprise determining a count of 100 or more hybridized nucleic acids. The method may comprise determining a count of 10000 or more hybridized nucleic acids based on the detection of the one or more nucleic acids. The method may comprise determining a count of 40000 or more hybridized nucleic acids. Detecting the one or more nucleic acids may comprise detecting 100 or more individual probes on the solid support. Detecting the one or more nucleic acids may comprise detecting 1000 or more individual probes on the solid support. Detecting the one or more nucleic acids may comprise detecting 10000 or more individual probes on the solid support. Detecting the one or more nucleic acids may comprise detecting 40000 or more individual probes on the solid support. Detecting the one or more nucleic acids may comprise detecting 50000 or more individual probes on the solid support. The plurality of nucleic acids may comprise one or more molecular species. The plurality of nucleic acids may comprise two or more molecular species. The plurality of nucleic acids may comprise 50000 or more nucleic acids. The plurality of probes may comprise 200 or more different probes. The plurality of probes may comprise 300 or more different probes. The plurality of probes may comprise 200 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The nucleic acids may be from a sample. The sample may be a blood or plasma sample. The sample may be a urine sample. The sample may be a stool sample.

In some instances, the disease or condition is pregnancy. For example, the methods and kits disclosed herein may be used in prenatal diagnosis. The method may comprise (a) obtaining a sample from a pregnant subject; (b) hybridizing at least a portion of the sample from the pregnant subject to a solid support, wherein the portion of the sample has a concentration of less than 1 genome equivalent; (c) detecting one or more nucleic acids hybridized to the solid support; and (d) diagnosing, prognosing, and/or monitoring a status or outcome of a fetal disorder based on the detection of the one or more nucleic acids. The method may further comprise determining a count of one or more nucleic acids based on the detection of the one or more nucleic acids. The method may further comprise determining a count of 100 or more nucleic acids based on the detection of the one or more nucleic acids. The method may further comprise determining a count of 10000 or more nucleic acids based on the detection of the one or more nucleic acids. The method may further comprise determining a count of 40000 or more nucleic acids based on the detection of the one or more nucleic acids. Detecting the one or more nucleic acids may comprise detecting individual nucleic acids hybridized to the solid support. Detecting the one or more nucleic acids may comprise detecting a plurality of nucleic acids. The plurality of nucleic acids may comprise one or more molecular species. The plurality of nucleic acids may comprise two or more molecular species. The plurality of nucleic acids may comprise 50000 or more nucleic acids. The solid support may comprise a plurality of probes. The one or more nucleic acids may hybridize to one or more probes. The plurality of probes may comprise 150 or more probes. The plurality of probes may comprise 300 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The sample may be a blood or plasma sample. The sample may be a urine sample. The sample may be a stool sample. The methods and kits disclosed herein may comprise diagnosing a fetal condition in a pregnant subject. The methods and kits disclosed herein may comprise identifying fetal mutations or genetic abnormalities. The nucleic acids to be detected may be from a fetal cell or tissue. Alternatively, or additionally, the nucleic acids to be detected may be from the pregnant subject.

Further disclosed herein is a method comprising (a) obtaining a sample from a pregnant subject; (b) hybridizing at least a portion of the sample from the pregnant subject to a solid support, wherein the solid support comprises a plurality of probes; (c) detecting one or more individual probes of the plurality of probes on the solid support, thereby determining a count of one or more nucleic acids in the sample; and (d) diagnosing, prognosing, and/or monitoring a status or outcome of a fetal disorder based on the count of the one or more nucleic acids. (a) hybridizing a plurality of nucleic acids to a plurality of probes on a solid support to produce one or more hybridized nucleic acids, wherein the plurality of probes comprises 150 or more different probes; and (b) determining a count of the one or more hybridized nucleic acids by counting individual probes on the solid support. The method may comprise determining a count of 100 or more hybridized nucleic acids. The method may comprise determining a count of 10000 or more hybridized nucleic acids based on the detection of the one or more nucleic acids. The method may comprise determining a count of 40000 or more hybridized nucleic acids. Detecting the one or more nucleic acids may comprise detecting 100 or more individual probes on the solid support. Detecting the one or more nucleic acids may comprise detecting 1000 or more individual probes on the solid support. Detecting the one or more nucleic acids may comprise detecting 10000 or more individual probes on the solid support. Detecting the one or more nucleic acids may comprise detecting 40000 or more individual probes on the solid support. Detecting the one or more nucleic acids may comprise detecting 50000 or more individual probes on the solid support. The plurality of nucleic acids may comprise one or more molecular species. The plurality of nucleic acids may comprise two or more molecular species. The plurality of nucleic acids may comprise 50000 or more nucleic acids. The plurality of probes may comprise 200 or more different probes. The plurality of probes may comprise 300 or more different probes. The plurality of probes may comprise 200 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The nucleic acids may be from a sample. The sample may be a blood or plasma sample. The sample may be a urine sample. The sample may be a stool sample. The methods and kits disclosed herein may comprise diagnosing a fetal condition in a pregnant subject. The methods and kits disclosed herein may comprise identifying fetal mutations or genetic abnormalities. The nucleic acids to be detected may be from a fetal cell or tissue. Alternatively, or additionally, the nucleic acids to be detected may be from the pregnant subject.

In addition to noninvasive prenatal diagnosis, the present method may be used in situations when conventional intensity based copy number measurements are suboptimal. One example is aneuploidy detection of single cell, especially in preimplantation genetic diagnosis. Amplification bias of single cell whole genome amplification methods is notoriously known to obscure copy number and single nucleotide polymorphism (SNP) measurement. In such case, the genomic DNA of a single cell can first be fragmented and split into multiple aliquots, such that each aliquot has less than one haploid genome worth of materials. Each aliquot may then be amplified and hybridized to a genome-wide array. Because the measurement is digital instead of analog and SNP measurement may be homozygous, the amplification bias may be less of a problem for CNV and SNP measurement. Generally, the method may comprise (a) obtaining a sample comprising a plurality of nucleic acids from a subject; (b) contacting the plurality of nucleic acids with a solid support comprising a plurality of probes; and (c) determining a count of one or more nucleic acids in the plurality of nucleic acids by counting individual probes on the solid support. The method may comprise determining a count of 100 or more nucleic acids. The method may comprise determining a count of 10000 or more nucleic acids based on the detection of the one or more nucleic acids. The method may comprise determining a count of 40000 or more nucleic acids. Detecting the one or more nucleic acids may comprise detecting 100 or more individual probes on the solid support. Detecting the one or more nucleic acids may comprise detecting 1000 or more individual probes on the solid support. Detecting the one or more nucleic acids may comprise detecting 10000 or more individual probes on the solid support. Detecting the one or more nucleic acids may comprise detecting 40000 or more individual probes on the solid support. Detecting the one or more nucleic acids may comprise detecting 50000 or more individual probes on the solid support. The plurality of nucleic acids may comprise one or more molecular species. The plurality of nucleic acids may comprise two or more molecular species. The plurality of nucleic acids may comprise 50000 or more nucleic acids. The plurality of probes may comprise 200 or more different probes. The plurality of probes may comprise 300 or more different probes. The plurality of probes may comprise 200 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The nucleic acids may be from a sample. The sample may be a blood or plasma sample. The sample may be a urine sample. The sample may be a stool sample.

Alternatively, the method may comprise (a) hybridizing a plurality of nucleic acids to a plurality of probes on a solid support to produce one or more hybridized nucleic acids, wherein the plurality of probes comprises 150 or more different probes; and (b) determining a count of the one or more hybridized nucleic acids by counting individual probes on the solid support. The method may comprise determining a count of 100 or more hybridized nucleic acids. The method may comprise determining a count of 10000 or more hybridized nucleic acids based on the detection of the one or more nucleic acids. The method may comprise determining a count of 40000 or more hybridized nucleic acids. Detecting the one or more nucleic acids may comprise detecting 100 or more individual probes on the solid support. Detecting the one or more nucleic acids may comprise detecting 1000 or more individual probes on the solid support. Detecting the one or more nucleic acids may comprise detecting 10000 or more individual probes on the solid support. Detecting the one or more nucleic acids may comprise detecting 40000 or more individual probes on the solid support. Detecting the one or more nucleic acids may comprise detecting 50000 or more individual probes on the solid support. The plurality of nucleic acids may comprise one or more molecular species. The plurality of nucleic acids may comprise two or more molecular species. The plurality of nucleic acids may comprise 50000 or more nucleic acids. The plurality of probes may comprise 200 or more different probes. The plurality of probes may comprise 300 or more different probes. The plurality of probes may comprise 200 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The nucleic acids may be from a sample. The sample may be a blood or plasma sample. The sample may be a urine sample. The sample may be a stool sample.

The methods and kits disclosed herein may be used in the diagnosis, prediction or monitoring of autosomal trisomies (e.g., Trisomy 13, 15, 16, 18, 21, or 22). In some cases the trisomy may be associated with an increased chance of miscarriage (e.g., Trisomy 15, 16, or 22). In other cases, the trisomy that is detected is a liveborn trisomy that may indicate that an infant will be born with birth defects (e.g., Trisomy 13 (Patau Syndrome), Trisomy 18 (Edwards Syndrome), and Trisomy 21 (Down Syndrome)). The abnormality may also be of a sex chromosome (e.g., XXY (Klinefelter's Syndrome), XYY (Jacobs Syndrome), or XXX (Trisomy X). The molecule(s) to be labeled may be on one or more of the following chromosomes: 13, 18, 21, X, or Y. For example, the molecule is on chromosome 21 and/or on chromosome 18, and/or on chromosome 13.

Further fetal conditions that may be determined based on the methods and kits disclosed herein include monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g. XXXX, XXYY, XXXY, XYYY, XXXXX, XXXXY, XXXYY, XYYYY and XXYYY), monoploidy, triploidy (three of every chromosome, e.g. 69 chromosomes in humans), tetraploidy (four of every chromosome, e.g. 92 chromosomes in humans), pentaploidy and multiploidy.

Forensic scientists may use nucleic acids in various samples (e.g. blood, semen, skin, saliva, hair) found at a crime scene to identify the presence of an individual at the scene, such as a perpetrator. This process is formally termed DNA profiling, but may also be called “genetic fingerprinting.” For example, DNA profiling comprises measuring and comparing the lengths of variable sections of repetitive DNA, such as short tandem repeats and minisatellites, in various samples and people. This method is usually an extremely reliable technique for matching a DNA sample from a person with DNA in a sample found at the crime scene. However, identification may be complicated if the scene is contaminated with DNA from several people. In this instance, as well as in other forensic applications, it may be advantageous to obtain absolute quantification of nucleic acids from a single cell or small number of cells.

Further disclosed herein is a method of forensic analysis comprising (a) obtaining a sample containing one or more nucleic acids; (b) hybridizing at least a portion of the sample to a solid support, wherein the concentration of the portion of the sample is less than 1 genome equivalent; and (c) detecting one or more nucleic acids hybridized to the solid support. The method may further comprise determining a count of one or more nucleic acids based on the detection of the one or more nucleic acids. The method may further comprise determining a count of 100 or more nucleic acids based on the detection of the one or more nucleic acids. The method may further comprise determining a count of 10000 or more nucleic acids based on the detection of the one or more nucleic acids. The method may further comprise determining a count of 40000 or more nucleic acids based on the detection of the one or more nucleic acids. Detecting the one or more nucleic acids may comprise detecting individual nucleic acids hybridized to the solid support. Detecting the one or more nucleic acids may comprise detecting a plurality of nucleic acids. The plurality of nucleic acids may comprise one or more molecular species. The plurality of nucleic acids may comprise two or more molecular species. The plurality of nucleic acids may comprise 50000 or more nucleic acids. The solid support may comprise a plurality of probes. The one or more nucleic acids may hybridize to one or more probes. The plurality of probes may comprise 150 or more probes. The plurality of probes may comprise 300 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label.

Further disclosed herein is a method of forensic analysis comprising (a) hybridizing a plurality of nucleic acids from a sample to one or more probes on an array to produce one or more hybridized nucleic acids, wherein the plurality of nucleic acids may comprise one or more molecular species; (b) scanning the array to obtain a detection signal for the one or more probes; (c) converting the detection signal to a digital signal; and (d) determining a count of the one or more hybridized nucleic acids based on a count of the digital signal. Further disclosed herein is a method of forensic analysis comprising (a) obtaining a sample from a subject, wherein the sample comprises a plurality of nucleic acids; (b) hybridizing the plurality of nucleic acids to one or more probes on a solid support to produce one or more hybridized nucleic acids, wherein the plurality of nucleic acids comprise one or more molecular species; and (c) determining a count of the one or more hybridized nucleic acids by counting individual probes on the solid support. Alternatively, the method of forensic analysis comprises (a) hybridizing a plurality of nucleic acids to a plurality of probes on a solid support to produce one or more hybridized nucleic acids, wherein the plurality of probes comprises 150 or more different probes; and (b) determining a count of the one or more hybridized nucleic acids by counting individual probes on the solid support. The method may comprise determining a count of 100 or more hybridized nucleic acids. The method may comprise determining a count of 10000 or more hybridized nucleic acids based on the detection of the one or more nucleic acids. The method may comprise determining a count of 40000 or more hybridized nucleic acids. Detecting the one or more nucleic acids may comprise detecting 100 or more individual probes on the solid support. Detecting the one or more nucleic acids may comprise detecting 1000 or more individual probes on the solid support. Detecting the one or more nucleic acids may comprise detecting 10000 or more individual probes on the solid support. Detecting the one or more nucleic acids may comprise detecting 40000 or more individual probes on the solid support. Detecting the one or more nucleic acids may comprise detecting 50000 or more individual probes on the solid support. The plurality of nucleic acids may comprise one or more molecular species. The plurality of nucleic acids may comprise two or more molecular species. The plurality of nucleic acids may comprise 50000 or more nucleic acids. The plurality of probes may comprise 200 or more different probes. The plurality of probes may comprise 300 or more different probes. The plurality of probes may comprise 200 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The nucleic acids may be from a sample. The sample may be a blood or plasma sample. The sample may be a urine sample. The sample may be a stool sample. The sample may be from a subject. The subject may suffer from a disease or condition. The subject may be healthy. The subject may be pregnant. A quantity of the plurality of nucleic acids in the sample may be less than 1 genome equivalent. A quantity of the plurality of nucleic acids in the sample may be less than 1 haploid genome equivalent.

Further disclosed herein is a method of low template DNA analysis comprising (a) obtaining a sample containing one or more nucleic acids; (b) hybridizing at least a portion of the sample to a solid support, wherein the concentration of the portion of the sample is less than 1 genome equivalent; and (c) detecting one or more nucleic acids hybridized to the solid support. Alternatively, the method comprises (a) hybridizing a plurality of nucleic acids from a sample to one or more probes on an array to produce one or more hybridized nucleic acids, wherein the plurality of nucleic acids may comprise one or more molecular species; (b) scanning the array to obtain a detection signal for the one or more probes; (c) converting the detection signal to a digital signal; and (d) determining a count of the one or more hybridized nucleic acids based on a count of the digital signal. The method may further comprise determining a count of one or more nucleic acids based on the detection of the one or more nucleic acids. The method may further comprise determining a count of 100 or more nucleic acids based on the detection of the one or more nucleic acids. The method may further comprise determining a count of 10000 or more nucleic acids based on the detection of the one or more nucleic acids. The method may further comprise determining a count of 40000 or more nucleic acids based on the detection of the one or more nucleic acids. Detecting the one or more nucleic acids may comprise detecting individual nucleic acids hybridized to the solid support. Detecting the one or more nucleic acids may comprise detecting a plurality of nucleic acids. The plurality of nucleic acids may comprise one or more molecular species. The plurality of nucleic acids may comprise two or more molecular species. The plurality of nucleic acids may comprise 50000 or more nucleic acids. The solid support may comprise a plurality of probes. The one or more nucleic acids may hybridize to one or more probes. The plurality of probes may comprise 150 or more probes. The plurality of probes may comprise 300 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label.

Further disclosed herein is a method of low template DNA analysis comprising (a) obtaining a sample from a subject, wherein the sample comprises a plurality of nucleic acids; (b) hybridizing the plurality of nucleic acids to one or more probes on a solid support to produce one or more hybridized nucleic acids, wherein the plurality of nucleic acids comprise one or more molecular species; and (c) determining a count of the one or more hybridized nucleic acids by counting individual probes on the solid support. Alternatively, the method comprises (a) hybridizing a plurality of nucleic acids to a plurality of probes on a solid support to produce one or more hybridized nucleic acids, wherein the plurality of probes comprises 150 or more different probes; and (b) determining a count of the one or more hybridized nucleic acids by counting individual probes on the solid support. The method may comprise determining a count of 100 or more hybridized nucleic acids. The method may comprise determining a count of 10000 or more hybridized nucleic acids based on the detection of the one or more nucleic acids. The method may comprise determining a count of 40000 or more hybridized nucleic acids. Detecting the one or more nucleic acids may comprise detecting 100 or more individual probes on the solid support. Detecting the one or more nucleic acids may comprise detecting 1000 or more individual probes on the solid support. Detecting the one or more nucleic acids may comprise detecting 10000 or more individual probes on the solid support. Detecting the one or more nucleic acids may comprise detecting 40000 or more individual probes on the solid support. Detecting the one or more nucleic acids may comprise detecting 50000 or more individual probes on the solid support. The plurality of nucleic acids may comprise one or more molecular species. The plurality of nucleic acids may comprise two or more molecular species. The plurality of nucleic acids may comprise 50000 or more nucleic acids. The plurality of probes may comprise 200 or more different probes. The plurality of probes may comprise 300 or more different probes. The plurality of probes may comprise 200 or more probes. The plurality of probes may be between about 10 to about 100 nucleotides in length. The plurality of probes may be between about 20 to about 60 nucleotides in length. The plurality of probes may hybridize to one or more regions on the nucleic acids. The plurality of probes may hybridize to overlapping regions on the nucleic acids. The plurality of probes may hybridize to non-overlapping regions on the nucleic acids. The plurality of probes may comprise two or more tiling probes (e.g., overlapping probes). At least 10% of probes in the plurality of probes may be tiling probes. The plurality of probes may comprise two or more different probes. At least about 10% of probes in the plurality of probes may be different. The probes may comprise a detectable label. The probes may comprise a target binding region. The probes may be targeted to a chromosome. The probes may be targeted to a gene. The solid support may be an array. The array may be a microarray. The microarray may be a tiling array. The nucleic acid may be DNA. The nucleic acid may be RNA. The nucleic acid may be cell-free nucleic acid. The nucleic acid may originate from a cell. The nucleic acid may be a circulating nucleic acid. The nucleic acid may be between about 20 to about 200 basepairs in length. The method may further comprise shearing the one or more nucleic acids. The method may further comprise end-polishing the one or more nucleic acids. The method may further comprise polyA tailing the one or more nucleic acids. The method may further comprise ligating one or more adaptors to the one or more nucleic acids. The adaptors may comprise a universal primer region. The adaptors may comprise a sequencing primer region. The method may further comprise determining a concentration of the nucleic acids. The method may further comprise amplifying the one or more nucleic acids. The method may further comprise fragmenting the one or more nucleic acids. The method may further comprise labeling the one or more nucleic acids with a label. The label may be a detectable label. The nucleic acids may be from a sample. The sample may be a blood or plasma sample. The sample may be a urine sample. The sample may be a stool sample. The sample may be from a subject. The subject may suffer from a disease or condition. The subject may be healthy. The subject may be pregnant.

### EXAMPLES

The following illustrative examples are representative of embodiments of the software applications, systems, and methods described herein and are not meant to be limiting in any way.

### Example 1

### Noninvasive Prenatal Diagnosis of Fetal Aneuploidy

An exemplary method for noninvasive prenatal diagnosis using array-based digital measurements is described in this example.

Part I: Nucleic Acid Sample Preparation

Step 1. Extract cell-free DNA from maternal blood.

Step 2. Add priming sites on both ends of the cell-free DNA fragments to facilitate universal PCR. Methods to add priming sites to both ends of cell-free DNA fragments may comprise (a) end polishing of cell-free DNA or fragmented cell-free DNA, followed by A-tailing, and ligation of common adaptors; (b) end polishing of cell-free DNA or fragmented cell-free DNA, followed by blunt end ligation of common adaptors; and/or (c) restriction digest of fragments with a 4 by cutter and ligation of common adaptor on the sticky end and another adaptor via blunt end ligation.

Part II: Dilution of Nucleic Acid Sample

Step 1. Quantitate DNA fragments that have properly ligated adaptors using methods such as real-time PCR or digital PCR.

Step 2. Calculate the number of DNA fragments required that correspond to less than 1 haploid genome. For instance, in the case that adaptors are ligated to both ends of end polished cell-free DNA fragments, given that the size distribution of cell-free DNA fragments is tight with an average of 160 bp, the approximate number of fragments that corresponds to one haploid genome is 3×10⁹ (size of haploid human genome) divided by 160,e.g., 18.75 million. For instance, 20% of a haploid genome would be equivalent to approximately 3.75 million fragments.

Step 3. Retrieve aliquots of the quantitated sample, with the amount of each aliquot containing fragments that correspond to less than 1 human haploid genome.

Step 4. Conduct PCR of each aliquot using primers complementary to the adaptor sequences, such that the PCR products of each aliquot are derived from less than 1 human haploid genome. The PCR primers can be labeled on the 5′ end with a molecule (e.g. fluorophore, biotin etc.) to facilitate subsequent signal detection after hybridization. In addition, the molecule attached to the primer can be different for different aliquots to differentiate hybridization events across PCR products of multiple aliquots within the same microarray or bead pool. The amplification conditions should be carefully controlled in order to suppress as much bias as possible (e.g. GC bias), for instance, by using engineered polymerases, or incorporating PCR additives.

Part III. Microarray Hybridization

Hybridize the PCR products of each aliquot onto a microarray or a bead pool with library of probes specific to the human genome. Because PCR products from each aliquot are derived from less than 1 haploid genome, only a subset of the probes would yield signal. By counting the number of positive probes and comparing the counts across different parts of the genome, one can obtain the relative abundance of a region.

Previous work on digital PCR and shotgun sequencing for noninvasive fetal aneuploidy detection determined the counting requirement scales with the fraction of fetal DNA in maternal blood. For shotgun sequencing for trisomy 21 detection, it was previously shown that 5 million unique reads over the entire repeat masked genome, or 60 k of chromosome 21 reads, are required. For the present method, the number of probes should be larger than the counting requirement to enable digital counting. The total length of repeat masked chromosome 21 is ˜18 Mb, representing ˜114 k non-overlapping 160 bp region. By hybridizing PCR products of ˜0.5 haploid genome equivalent worth of cell-free DNA on a set of probes tiled across chromosome 21, one can achieve ˜60 k counts of chromosome 21.

To reduce hybridization noise, multiple aliquots of further diluted cell-free DNA can be used. For instance, hybridizations of PCR products derived from two different aliquots of ˜0.25 haploid genome worth of materials would meet the same counting requirement. The PCR products can be labeled with different dyes and be hybridized to the same probe library.

Because the length of cell-free DNA (˜160 bp) is much longer than the probe length (usually 25-60mer), two different fragments, defined by having different ends, but with overlap, would hybridize to the same probe. To increase the available count per aliquot, cell-free DNA can be digested randomly to generate shorter fragments (e.g. ˜60 bp) before adaptor ligation and universal PCR. This would increase the available count by more than 2 fold (˜18 Mb/160 bp vs. ˜180 Mb/60 bp).

Another method to increase the available count per aliquot is to incorporate reaction to assay the end of the hybridized fragments. One such assay is single base extension. To differentiate one vs. two unique fragments hybridizing to the same probe, a set of ddNTPs, each with a different dye, together with a primer complementary to the adaptor, can be incorporated to the hybridized products. The presence of two different bases indicates overlapping fragments.

For prenatal diagnosis, the probe library does not have to cover the entire genome, but would contain probes specific for chromosomes and sub-chromosomal regions that are highly prone to aneuploidy (e.g. 21, 18, 13, X, Y). If only one high-risk chromosome is included in the probe library, the library should also contain probes specific for a chromosome that is not likely to be associated with aneuploidy to serve as reference.

Cross hybridization noise can be differentiated from signal based on expected fragment size. For instance, in the case of undigested cell-free DNA fragments (e.g., adaptors attached on end-polished cell-free DNA fragments) and the use of tiling probes, given the cell-free DNA has an average size of 160 bp, one expects hybridization of a set of probes that span a total length of ˜160 bp (e.g. for a tiling array with 25mer probe and 9nt spacing, one expects 4-5 contiguous probes to light up for a 160 bp fragment). Random cross-hybridization events would most likely result in signal from individual probes scattered across the genome.

Cross hybridization noise can also be differentiated from signal by detecting ligation event upon legitimate hybridization.

The counting requirement for aneuploidy detection depends on fetal DNA fraction. Because the main source of fetal DNA is the placenta, which has been shown to be hypomethylated compared to whole blood, instead of ligating adaptors to all cell-free DNA fragments, one can specifically add adaptors to unmethylated cell-free DNA fragments. Methylation sensitive restriction enzymes (e.g. HpaII, Hin6I, AciI) can be used to generate sticky ends specifically on unmethylated fragments. Adaptors can be ligated to the sticky ends, thus only unmethylated fragments would be counted.

### Example 2

### Noninvasive Prenatal Diagnosis by Single Hybridization of Barcoded Molecules Originated from Multiple Genomic Loci

Presented here is a method to count multiple loci across the genome simultaneously using barcoding strategy in a single hybridization. Instead of using the same set of barcodes for each genomic locus, which requires one hybridization per locus, each genomic locus is assigned its own set of barcodes, such that each molecule to be counted in this multiplex measurement receives a unique barcode.

For specific application of noninvasive fetal aneuploidy detection:

Synthesize a set of 100 k to 200 k probes per chromosome. At least two chromosomes are measured at a time—one of high-risk of aneuploidy (e.g. chr21) and one of low-risk of aneuploidy (e.g. chr12) to serve as reference. Each probe consists of three parts: one part carries a universal priming sequence, one part targets a genomic region and one part carries a barcode (˜20nt) that is a non-human sequence. Each of the 100 k to 200 k probes has a different barcode. The probes can each target a different genomic loci or a small set of genomic loci (for instance, a probe set of 100 k probes targets 1000 non-repetitive regions on a chromosome; for each region, there are 100 different kind of probes, each kind bears a unique barcode; the barcode of each of the 100 k probe is different).

The probe may be molecular inversion probe, in which the part of the probe targeting the genomic region is divided into two subparts. Upon hybridization, polymerase and ligase activity circularizes the probe. Un-hybridized probes are digested by exonuclease.

The probe set is hybridized to cell-free DNA extracted from maternal blood. The amount of cell-free DNA has to be adjusted depending on the barcode space at each genomic locus. For the example above, barcode space is 100 (100 different barcodes per locus). The number of genome equivalent of cell-free DNA is <100.

The hybridized probes are amplified by PCR via universal priming sequence, and hybridized to a custom made array with probes complementary to the initial probe set. The count of a specific locus is determined by the number of positive barcodes specific to that locus.

### Example 3

### Non-Invasive Prenatal Diagnostics

Sample preparation, array hybridization and detection were performed as follows: DNA was sheared into 100-200 bp fragments by Covaris. The sheared DNA was end-polished. A polyadenylated tail was added to the end-polished DNA by A-tailing. Adaptors were ligated to the polyA-tailed DNA. Quantitative PCR was performed to estimate the concentration of the ligated molecules. Based on the concentration of the ligated molecules, 1-10% of a haploid genome equivalent of ligated molecules was amplified.

For approximately 10% (or 0.1x) haploid genome, the following samples were tested:

10% (0.1x) haploid genome

| Sample Name | Description |
|---|---|
| 100% Trisomy 0.1x | 100% trisomy21 |
| 30% Trisomy 0.1x | 30% trisomy21 + 70% normal |
| 20% Trisomy 0.1x | 20% trisomy21 + 80% normal |
| 10% Trisomy 0.1x | 10% trisomy21 + 90% normal |
| Normal 0.1x | 100% normal |

For approximately 1% (or 0.01x) haploid genome, the following samples were tested:

1% (0.01x) haploid genome

| Sample Name | Description |
|---|---|
| 100% Trisomy 0.01x | 100% trisomy21 |
| 30% Trisomy 0.01x | 30% trisomy21 + 70% normal |
| 20% Trisomy 0.01x | 20% trisomy21 + 80% normal |
| Normal 0.01x | 100% normal |

20-30 molecules of each of three controls were spiked into each sample before the first amplification. Two of the controls were on chromosome 22 and one control was on chromosome 21. The controls were made of 160 bp PCR products.

A second PCR was performed to generate up to ˜100 μg of materials, of which approximately 50% was genomic and approximately 50% was adaptor. The amplified molecules were fragmented with a Dnase. The fragmented molecules were ethanol precipated. The ethanol precipated molecules were DNA end labeled with a terminal transferase and biotinylated nucleotide.

The labeled molecules were hybridized to an Affymetrix chromosome 21/22 (Chr21/22) tiling array. The array with the hybridized molecules were washed, stained, and scanned. The array was stained with streptavidin-phycoerythrin (SAPE), followed by biotinylated anti-streptavidin antibody, and, lastly, SAPE again.

After the tiling arrays were scanned, positive hybridization events were called using model-based analysis of tiling arrays (MAT) as described in Johnson et al. (PNAS, 103(33):12457-12462). Generally, for each microarray, probe behavior was modeled based on probe sequence using multivariate linear regression. Noise from cross-hybridization was suppressed. A T-score was calculated for each probe. A trimmed mean T-score for each 160 bp region, which is the approximate length of sheared DNA or plasma DNA, was calculated by (1) sorting T-scores of probes within a region; (2) ignoring the lower and upper 10% of T-scores; and (3) calculating the mean of the remaining T-scores. Regions with 6 or fewer probes were ignored. On average, each region should have 10-11 probes based on the design of the tiling array. A MATscore was calculated by the following equation: MATscore=sqrt(number of probes)*trimmed mean T-score.

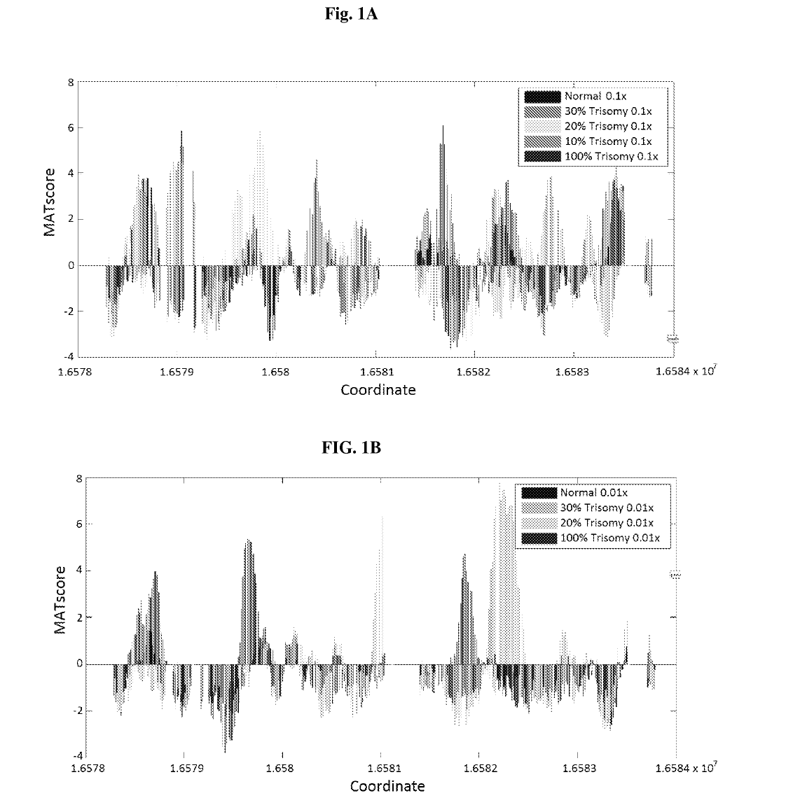

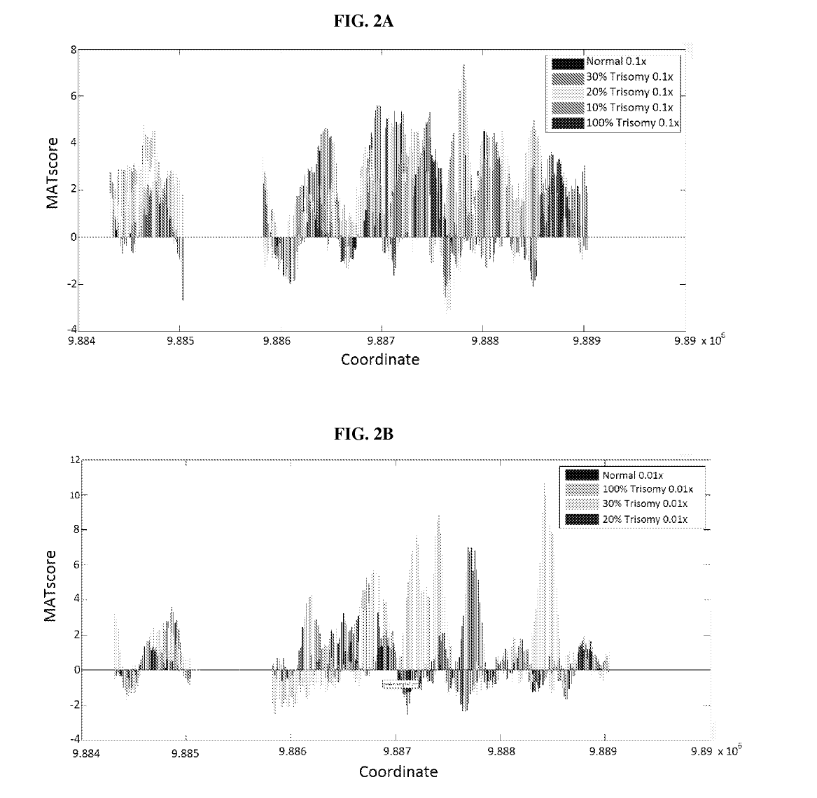

FIG. 1A-B show bar graphs of the MATscore for genomic coordinates 1.6578-1.6584×10⁷ at 0.1x haploid genome (FIG. 1A) and 0.01x haploid genome (FIG. 1B). FIG. 2A-B show bar graphs of the MATscore for genomic coordinates 9.884-9.89×10⁶ at 0.1x haploid genome (FIG. 2A) and 0.01x haploid genome (FIG. 2B). For FIG. 1-2, each bar corresponds to a MATscore of a probe. At higher concentrations, the peaks of the different samples may overlap, as shown in FIGS. 1A and 2A. At lower concentrations, the peaks of the different samples should have little overlap, as shown in FIGS. 1B and 2B.

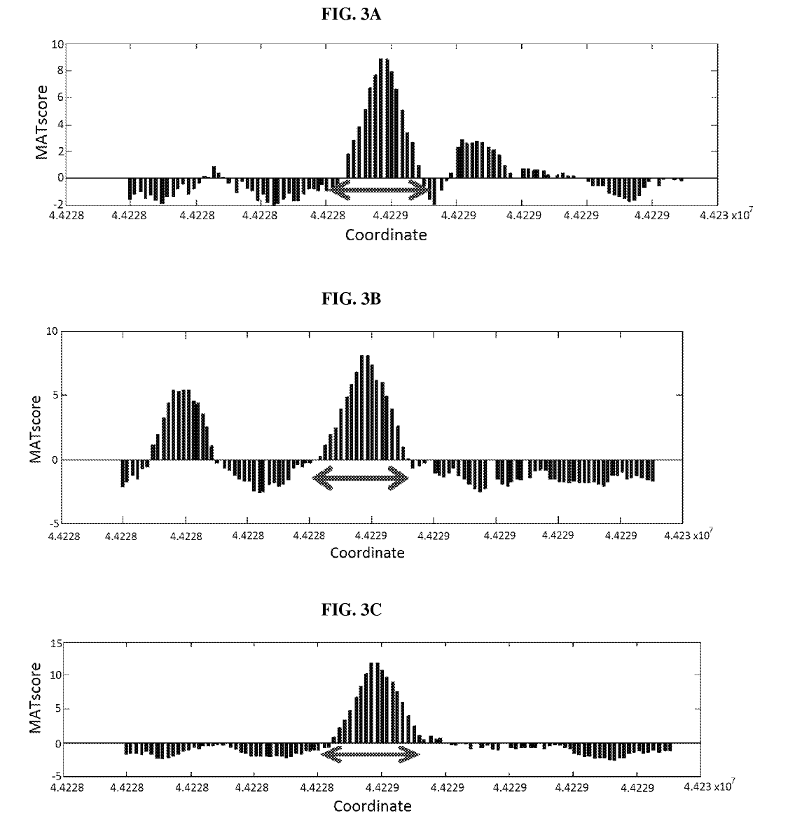

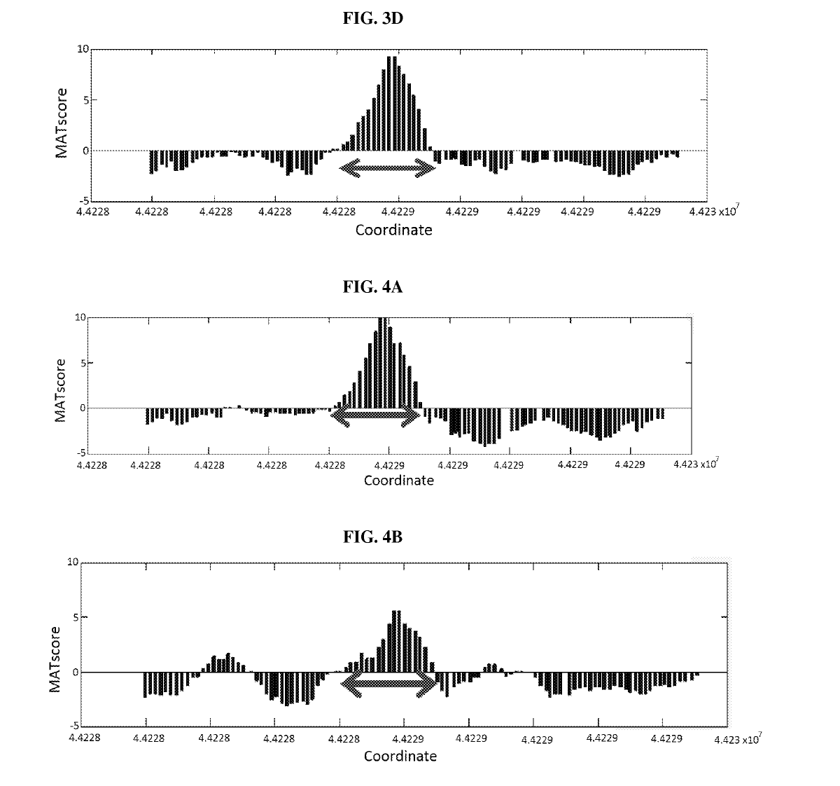

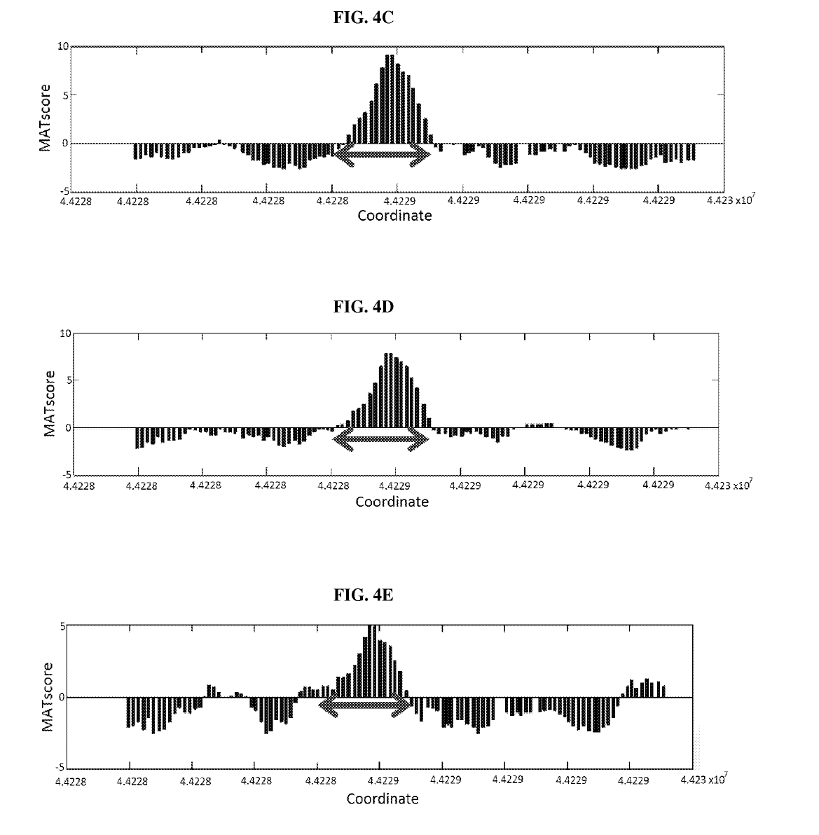

FIG. 3-4 show bar graphs of the MATscore for genomic coordinates corresponding 4.228-4.23×10⁷ at 0.01x haploid genome (FIG. 3A-D) and 0.1x haploid genome (FIG. 4A-E). For FIG. 3-4, the double headed arrows refer to the probes covering the genomic region of the first spiked in control. FIG. 3A shows the MATscore for the Normal 0.01x sample. FIG. 3B shows the MATscore for the 100% Trisomy 0.01x sample. FIG. 3C shows the MATscore for the 30% Trisomy 0.01x sample. FIG. 3D shows the MATscore for the 20% Trisomy 0.01x sample. FIG. 4A shows the MATscore for the Normal 0.1x sample. FIG. 4B shows the MATscore for the 100% Trisomy 0.1x sample. FIG. 4C shows the MATscore for the 30% Trisomy 0.1x sample. FIG. 4D shows the MATscore for the 20% Trisomy 0.1x sample. FIG. 4E shows the MATscore for the 10% Trisomy 0.1x sample.

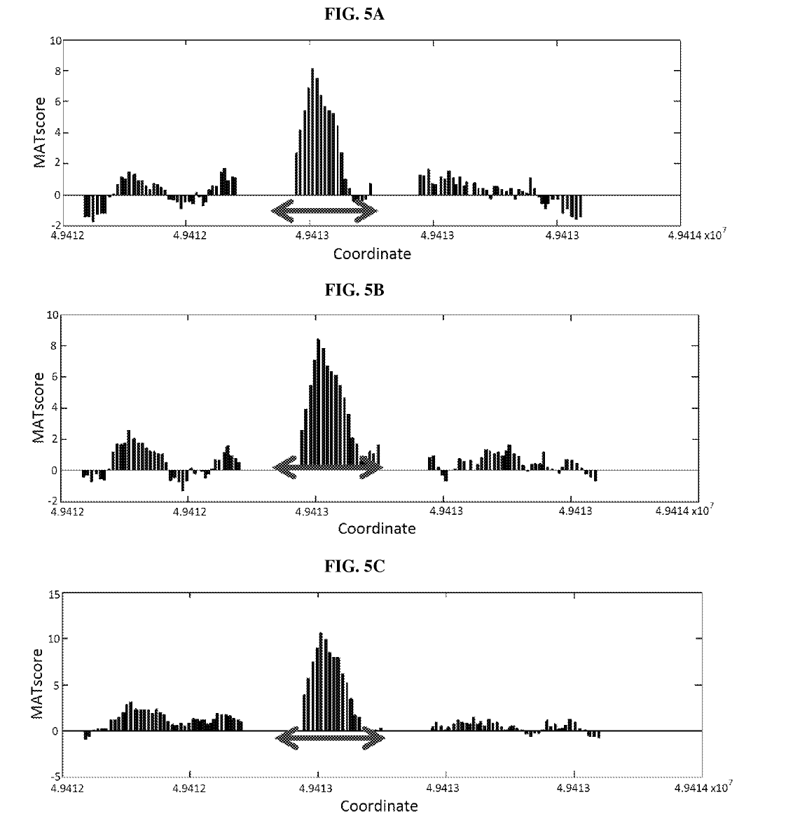

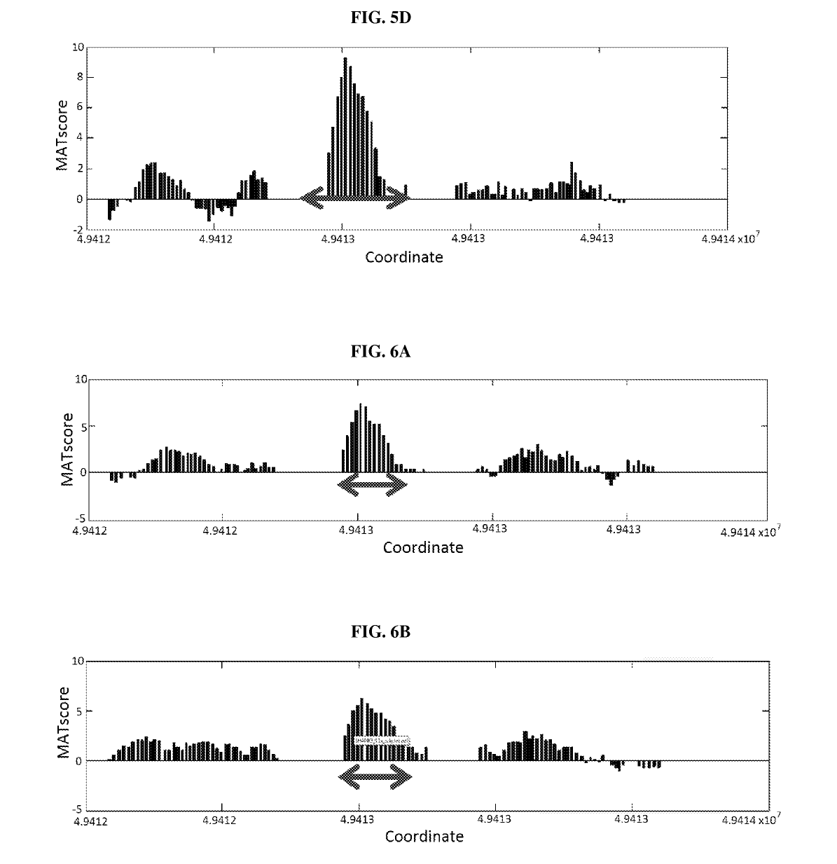

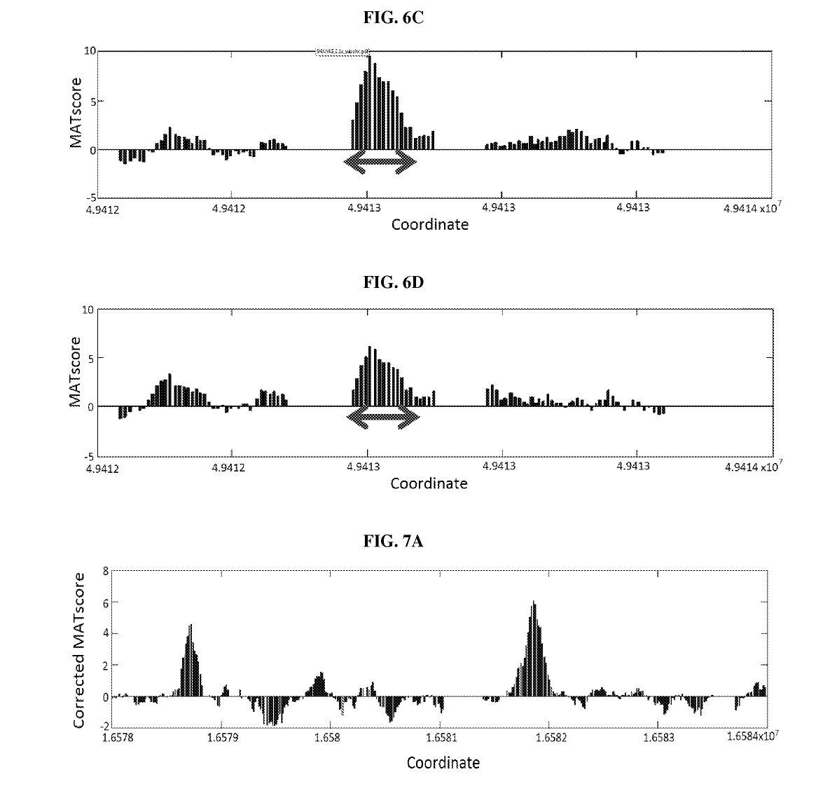

FIG. 5-6 show bar graphs of the MATscore for genomic coordinates corresponding 4.912-4.914×10⁷ at 0.01x haploid genome (FIG. 5A-D) and 0.1x haploid genome (FIG. 6A-D). For FIG. 5-6, the double headed arrows refer to the probes covering the genomic region of the second spiked in control. FIG. 5A shows the MATscore for the Normal 0.01x sample. FIG. 5B shows the MATscore for the 100% Trisomy 0.01x sample. FIG. 5C shows the MATscore for the 30% Trisomy 0.01x sample. FIG. 5D shows the MATscore for the 20% Trisomy 0.01x sample. FIG. 6A shows the MATscore for the Normal 0.1x sample. FIG. 6B shows the MATscore for the 100% Trisomy 0.1x sample. FIG. 6C shows the MATscore for the 30% Trisomy 0.1x sample. FIG. 6D shows the MATscore for the 20% Trisomy 0.1x sample.

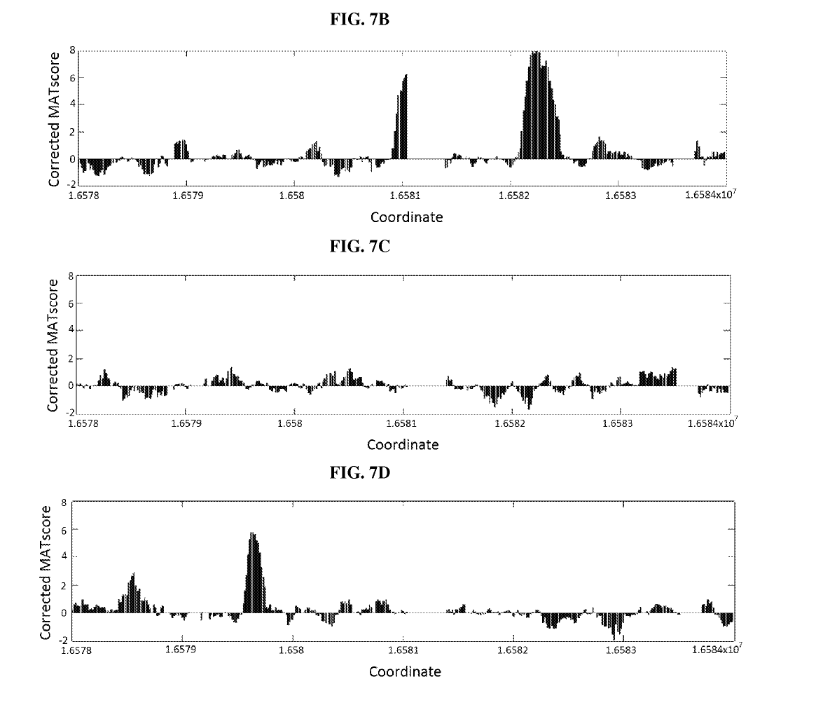

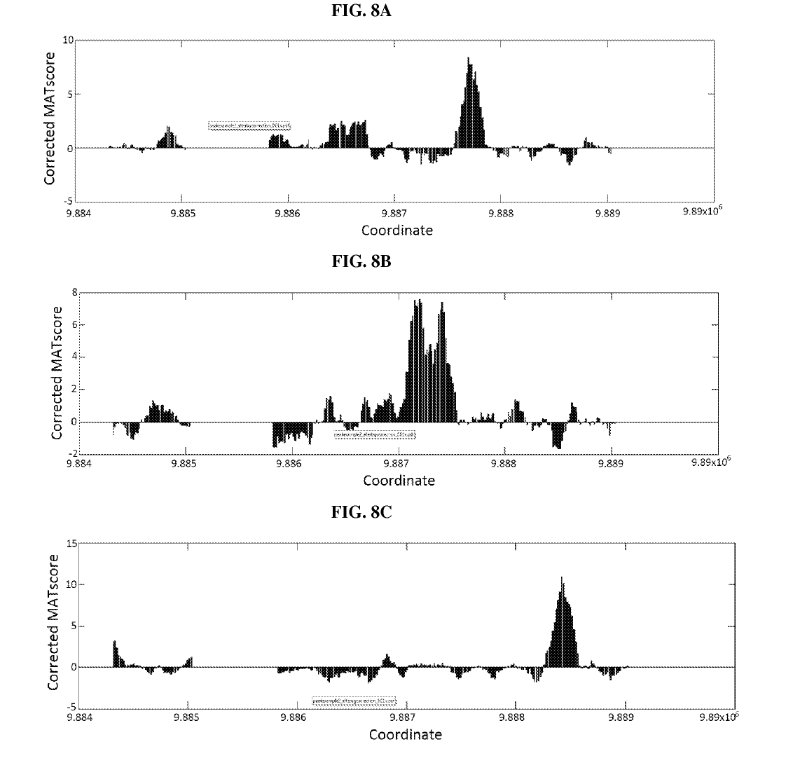

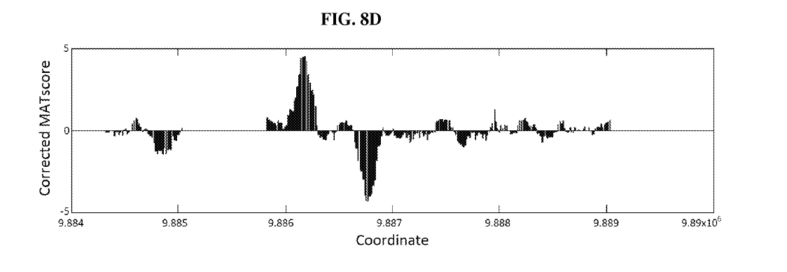

FIG. 1-6 show the MATscore before background substraction. In some genomic regions, the distribution of peaks was very similar across the different samples tested. The similarity of the distribution may be due to systematic artifact. To remove such artifact, at each concentration (0.1x and 0.01x), the median MATscore of each probe was calculated across the samples. For each sample, the corrected MATscore of each probe was computed as the difference between the original MATscore and the median MATscore. FIG. 7A-D show bar graphs of the corrected MATscore for genomic coordinates 1.6578-1.6584×10⁷ at 0.01x haploid genome. FIG. 7A shows the corrected MATscore for the Normal 0.01x sample. FIG. 7B shows the corrected MATscore for the 100% Trisomy 0.01x sample. FIG. 7C shows the corrected MATscore for the 30% Trisomy 0.01x sample. FIG. 7D shows the corrected MATscore for the 20% Trisomy 0.01x sample. FIG. 8A-D show bar graphs of the corrected MATscore for genomic coordinates 9.884-9.89×10⁶ at 0.01x haploid genome. FIG. 8A shows the corrected MATscore for the Normal 0.01x sample. FIG. 8B shows the corrected MATscore for the 100% Trisomy 0.01x sample. FIG. 8C shows the corrected MATscore for the 30% Trisomy 0.01x sample. FIG. 8D shows the corrected MATscore for the 20% Trisomy 0.01x sample.

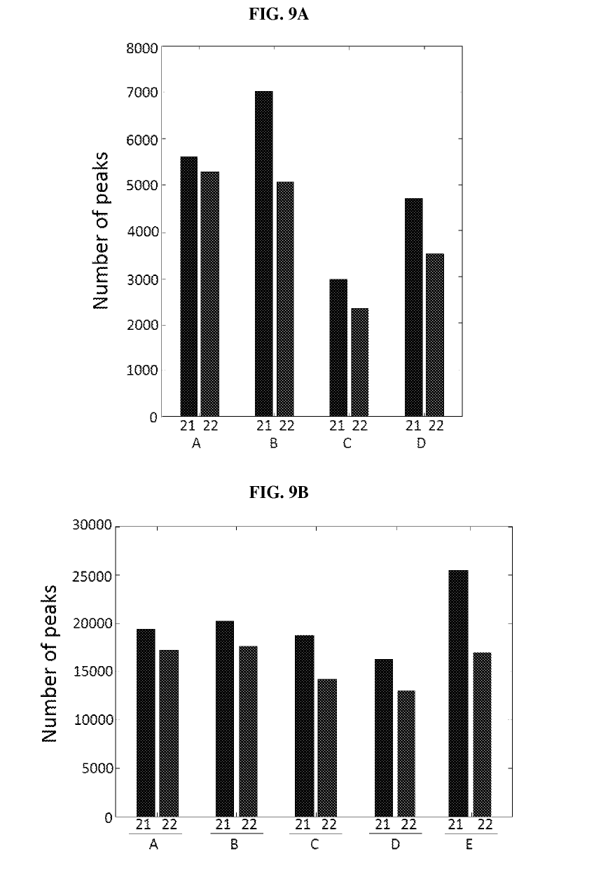

Hybridization events were counted as follows. The location of peaks across the chromosomes were determined by using an off the shelf peak finding algorithm. Chromosomes 21 and 22 were divided into fixed windows (e.g. 100 kb, 500 kb, 1 Mb). The number of peaks within each window were counted. FIG. 9A-B show bar graphs of the number of peaks over an entire chromosome (chromosomes 21 and 22) at 0.01x haploid genome (FIG. 9A) and 0.1x haploid genome (FIG. 9B). For FIG. 9A-9B, the numbers 21 and 22 on the x-axis refer to chromosome 21 and 22, respectively. On the x-axis of FIG. 9A, “A” refers to the Normal sample; “B” refers to the 100% Trisomy sample; “C” refers to the 30% Trisomy sample; and “D” refers to the 20% Trisomy sample. On the x-axis of FIG. 9B, “A” refers to the Normal sample; “B” refers to the 100% Trisomy sample; “C” refers to the 30% Trisomy sample; “D” refers to the 20% Trisomy sample; and “E” refers to the 10% Trisomy sample. A few thousand molecules on non-repetitive regions of chromosome 22 were observed for the 0.01x haploid genome samples (see FIG. 9A). A few tens of thousands of molecules on non-repetitive regions of chromosome 22 were observed for the 0.1x haploid genome samples (see FIG. 9B).

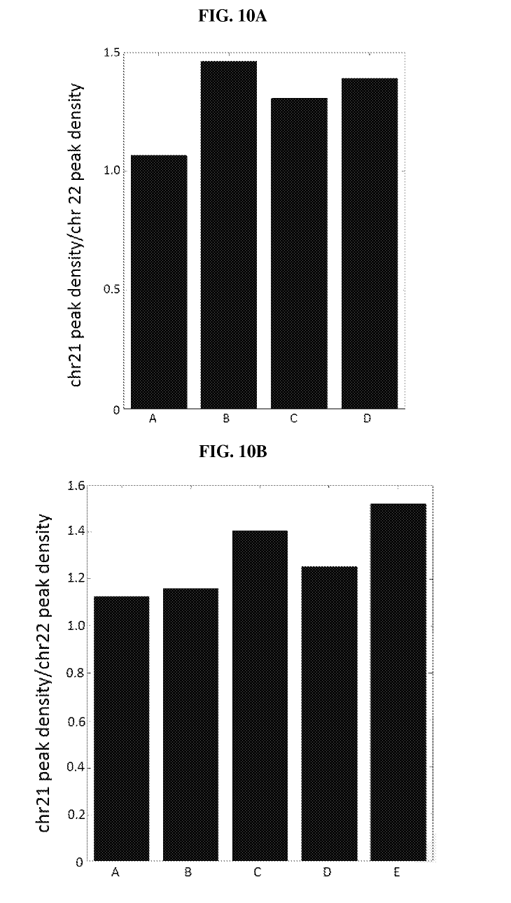

The median number of peaks per window for each of chromosomes 21 and 22 were calculated. A ratio of peak density of chromosome 21 to peak density of chromosome 22 was calculated. Table 1 shows the expected and actual ratios of peak density between chromosome 21 and 22 for the 0.01x and 0.1x haploid genome concentrations. A graph of the peak ratios for chromosome 21/22 for the 0.01x haploid concentration is shown in FIG. 10A. On the x-axis of FIG. 10A, “A” refers to the Normal sample; “B” refers to the 100% Trisomy sample; “C” refers to the 30% Trisomy sample; and “D” refers to the 20% Trisomy sample. A graph of the peak ratios for chromosome 21/22 for the 0.1x haploid concentration is shown in FIG. 10B. On the x-axis of FIG. 10B, “A” refers to the Normal sample; “B” refers to the 100% Trisomy sample; “C” refers to the 30% Trisomy sample; “D” refers to the 20% Trisomy sample; and “E” refers to the 10% Trisomy sample.

TABLE 1
Peak Ratio of Chr 21/22

| | Expected Ratio | 0.01x Actual Ratio | 0.1x Actual Ratio |
|---|---|---|---|
| Normal | 1.0 | 1.067 | 1.1276 |
| 100% Trisomy | 1.5 | 1.4652 | 1.5211 |
| 30% Trisomy | 1.15 | 1.3078 | 1.2541 |
| 20% Trisomy | 1.10 | 1.3919 | 1.405 |
| 10% Trisomy | 1.05 | N/A | 1.1609 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Example 4

### Non-Invasive Prenatal Diagnostics

Part I: Nucleic Acid Sample Preparation

Step 1. Extract Cell free DNA from maternal blood.

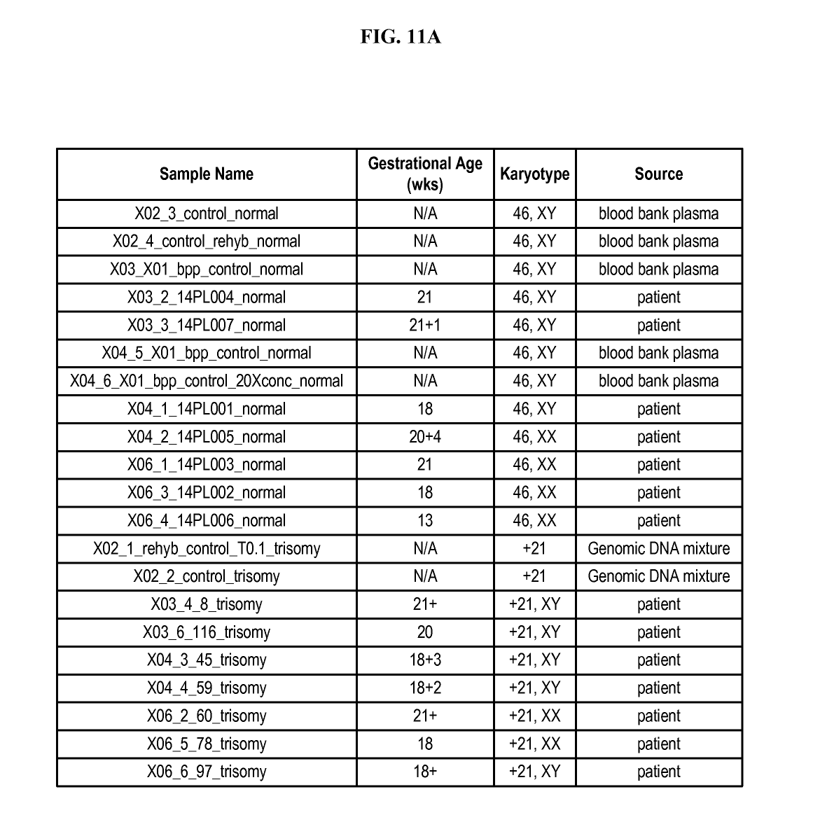

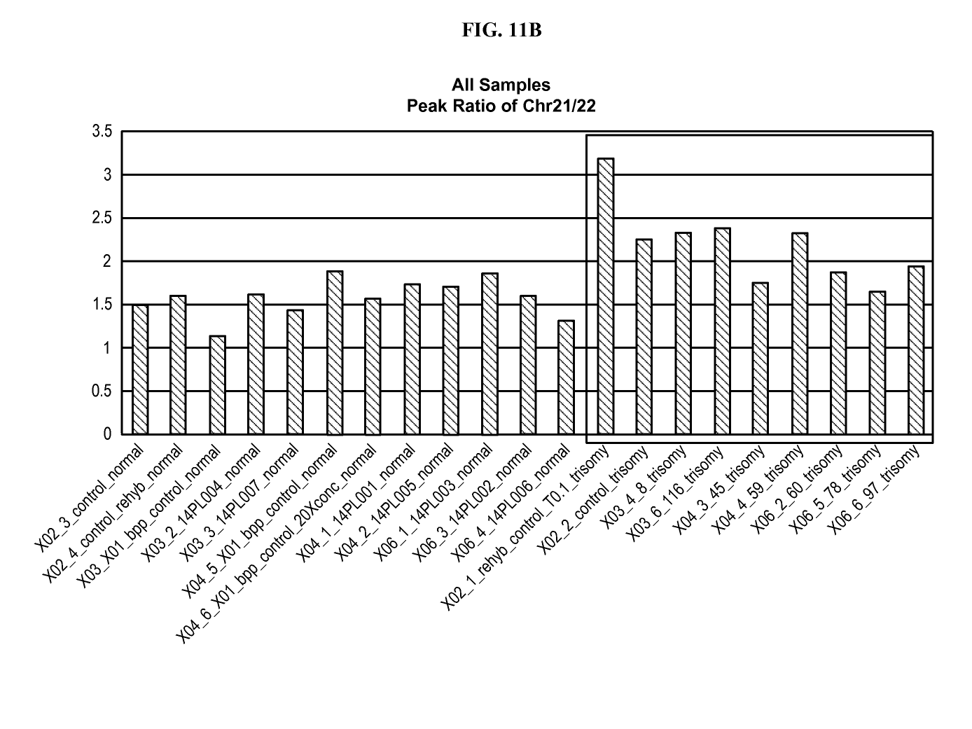

FIG. 11A shows a description of the samples processed and their descriptions.

Step 2. Add priming sites on both ends of the cell-free DNA fragments to facilitate universal PCR by end polishing the cell-free DNA, followed by A-tailing, and ligation of common adaptors;

Part II: Dilution of Nucleic Acid Sample

Step 1. Quantitate DNA fragments that have properly ligated adaptors using real-time PCR.

Step 2. Calculate the number of DNA fragments required that correspond to less than 1 haploid genome. For instance, in the case that adaptors are ligated to both ends of end polished cell-free DNA fragments, given that the size distribution of cell-free DNA fragments is tight with an average of 160 bp, the approximate number of fragments that corresponds to one haploid genome is 3×10⁹ (size of haploid human genome) divided by 160,e.g., 18.75 million. For instance, 20% of a haploid genome would be equivalent to approximately 3.75 million fragments.

Step 3. Retrieve aliquots of the quantitated sample, with the amount of each aliquot containing fragments that correspond to less than 1 human haploid genome.

Part III: Amplification of Cell-Free DNA

Step 1. Conduct PCR of each aliquot using primers complementary to the adaptor sequences, such that the PCR products of each aliquot are derived from less than 1 human haploid genome. The amplification conditions should be carefully controlled in order to suppress as much bias as possible (e.g. GC bias), for instance, by using engineered polymerases, or incorporating PCR additives.

Step 2. 20-30 molecules of each of three controls were spiked into each sample before the first amplification. Two of the controls were on chromosome 22 and one control was on chromosome 21. The controls were made of 160 bp PCR products.

Step 3. A second PCR was performed to generate up to ˜150 μg of materials. The amplified molecules were fragmented with a Dnase. The fragmented molecules were ethanol precipated. The ethanol precipated molecules were DNA end labeled with a terminal transferase and biotinylated nucleotide.

Part III: Hybridization

Step 1: The labeled molecules were hybridized to an Affymetrix chromosome 21/22 (Chr21/22) tiling array. The array with the hybridized molecules were washed, stained, and scanned. The array was stained with streptavidin-phycoerythrin (SAPE), followed by biotinylated anti-streptavidin antibody, and, lastly, SAPE again.

After the tiling arrays were scanned, positive hybridization events were called using model-based analysis of tiling arrays (MAT) as described in Johnson et al. (PNAS, 103(33):12457-12462). Generally, for each microarray, probe behavior was modeled based on probe sequence using multivariate linear regression. Noise from cross-hybridization was suppressed. A T-score was calculated for each probe. A trimmed mean T-score for each 160 bp region, which is the approximate length of sheared DNA or plasma DNA, was calculated by (1) sorting T-scores of probes within a region; (2) ignoring the lower and upper 10% of T-scores; and (3) calculating the mean of the remaining T-scores. Regions with 6 or fewer probes were ignored. On average, each region should have 10-11 probes based on the design of the tiling array. A MATscore was calculated by the following equation: MATscore=sqrt(number of probes)*trimmed mean T-score.

Hybridization events were counted as follows. The location of peaks across the chromosomes were determined by using an off the shelf peak finding algorithm. Chromosomes 21 and 22 were divided into fixed windows (e.g. 100 kb, 500 kb, 1 Mb). The number of peaks within each window were counted using a MATscore cutoff of 2.5.

The median number of peaks per window for each of chromosomes 21 and 22 were calculated. A ratio of peak density of chromosome 21 to peak density of chromosome 22 was calculated.

FIG. 11B shows a graph of the peak ratios for chromosome 21/22 using a MATscore cutoff of 2.5 for the samples described in FIG. 11A. The sample names are shown on the x-axis

1. A method comprising:
a. hybridizing a plurality of nucleic acids from a sample to one or more probes on an array to produce one or more hybridized nucleic acids, wherein the plurality of nucleic acids comprise one or more molecular species;
b. scanning the array to obtain a detection signal for the one or more probes;
c. converting the detection signal to a digital signal; and
d. determining a count of the one or more hybridized nucleic acids based on a count of the digital signal.

2. The method of claim 1, wherein the detection signal is an intensity signal.

3. The method of claim 2, wherein converting the detection signal to a digital signal comprises binary classification of the detection signal.

4. The method of claim 3, wherein the binary classification comprises classifying a probe as positive or negative.

5. The method of claim 4, wherein determining the count of the one or more hybridized nucleic acids comprises determining a count of probes that are classified as positive.

6. The method of claim 1, wherein converting the detection signal to a digital signal comprises modeling a behavior of the one or more probes based on the sequence of the probe.

7. The method of claim 6, wherein modeling the behavior of the one or more probes comprises applying an algorithm to the detection signal.

8. (canceled)

9. The method of claim 6, wherein modeling the behavior of the one or more probes comprises subtracting a probe-specific background from the detection signal of the probe.

10. The method of claim 6, wherein modeling the behavior of the one or more probes comprises clustering the probes into groups based on similarity of one or more features.

11. (canceled)

12. The method of claim 1, wherein the array is a microarray or a titling array.

13. (canceled)

14. The method of claim 1, wherein the array comprises a plurality of probes.

15. (canceled)

16. (canceled)

17. The method of claim 16, wherein the plurality of probes is capable of hybridizing to the one molecular species.

18. The method of claim 17, wherein the plurality of probes is capable of hybridizing to one or more regions on the one molecular species.

19. The method of claim 17, wherein the plurality of probes is capable of hybridizing to two or more regions on the one molecular species.

20. (canceled)

21. (canceled)

22. The method of claim 14, wherein at least two probes of the plurality of probes are different.

23. The method of claim 14, wherein at least two probes of the plurality of probes are less than 90% identical.

24. (canceled)

25. (canceled)

26. The method of claim 16, wherein the plurality of probes is capable of hybridizing to two or more molecular species.

27. The method of claim 14, wherein the plurality of probes comprises two or more sets of probes.

28. The method of claim 27, wherein the two or more sets of probes are capable of hybridizing to one molecular species.

29. The method of claim 27, wherein the two or more set of probes are capable of hybridizing to two or more molecular species.

30.-215. (canceled)
